SEQUENCE LISTING (4 sequences)

SEQ ID NO: 1 - protein, 34 aa, Severe acute respiratory syndrome coronavirus 2
SSNFGAISSVLNDILSRLDKVEAEVQIDRLITGR

SEQ ID NO: 2 - DNA, 3822 bp, Severe acute respiratory syndrome coronavirus 2
  source 1..3822: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 34 aa, synthetic construct
SSNCGAISSVLNDILSRLDKVEAEVQIDRLITCR

(19) United States
(12) Patent Application Publication
(10) Pub. No.: US 2023/0357325 A1
(43) Pub. Date: Nov. 9, 2023

(54) COMPOSITION AND METHOD TO STABILIZE CORONAVIRUS SPIKE GLYCOPROTEINS IN PRE-FUSION CONFORMATION

(71) Applicant: The Research Foundation for The State of University New York, Albany, NY (US)

(72) Inventor: Carlos Simmerling, Stony Brook, NY (US)

(21) Appl. No.: 18/314,769

(22) Filed: May 9, 2023

Related U.S. Application Data
(60) Provisional application No. 63/339,890, filed on May 9, 2022.

(51) Int. Cl. C07K 14/005

(52) U.S. Cl. CPC C07K 14/005; A61K 39/00

(57) ABSTRACT

Compositions include coronavirus S1/S2 prefusion spike proteins with specifically designed disulfide bond that “staple” together the central helix and a region of the spike known as HR1. By preventing HR1 from detaching from CH, the prefusion spike structure is stabilized without rigidification of the central helix or changes to its interaction with the receptor binding domain. This disulfide-stapled spike is more stable in the prefusion form, allowing for a stable vaccine without the need for the stabilizing mutations that are currently in use.

### CROSS REFERENCE TO RELATED APPLICATIONS

This application claims the benefit of priority under 35 U.S.C. § 119(e) to U.S. Provisional Application No. 63/339,890 filed May 9, 2022. The entire content of this application is incorporated herein by reference in its entirety.

### REFERENCE TO A SEQUENCE LISTING

This application incorporates by reference the Sequence Listing submitted in Computer Readable Form as file name Sequence Listing 162152-56101, created on May 9, 2023, and is 10 bytes in size.

### FIELD

The present disclosure relates in general to coronavirus spike proteins that include mutations to stabilize the 51 and S2 subunits in a prefusion state and their uses in producing vaccines.

### BACKGROUND

Coronaviruses (CoV) are enveloped viruses with a positive-stranded RNA genome. In December 2019, a novel coronavirus designated as 2019-nCoV (or SARS-CoV-2) appeared in Wuhan, China. SARS-CoV, MERS-CoV, and SARS-CoV-2 belong to the β-coronavirus genus and are highly pathogenic zoonotic viruses. In addition to these three highly pathogenic β-coronaviruses, four low-pathogenicity β-coronaviruses, HCoV-OC43, HCoVHKU1, HCoV-NL63 and HCoV-229E, are also endemic in humans.

The SARS-CoV-2 virus uses its spike proteins (S proteins) to bind host cellular receptor angiotensin-converting enzyme 2 (ACE2). Although the sequence and structure of the SARS-CoV-2 spike protein is a known (see, e.g., Daniel Wrapp et al., Cryo-EM structure of the 2019-nCoV spike in the prefusion conformation. Science 367, 1260-1263(2020). DOI:10.1126/science.abb2507) there remains a need for stabilized S proteins that could be used for identifying drug candidates and for stimulating an effective immune response to the S protein.

### SUMMARY

The disclosure provides engineered polypeptides derived or modified from the spike (S) glycoprotein of coronaviruses, including SARS-CoV, MERS-CoV and SARS-CoV-2. The polypeptides comprise S sequences with modifications that stabilize the pre-fusion S structure relative to the wild-type soluble S protein sequence of coronaviruses. The modifications include substitutions in the spike proteins which allow for the formation of a disulfide bridge that stabilizes the coronavirus S protein or a portion thereof (e.g., peptide sequence comprising the S1/S2) in the pre-fusion S structure.

Accordingly, in certain aspects, a coronavirus spike protein comprises an F to C (Phe to Cys) substitution and a G to C (Gly to Cys) substitution at positions corresponding to F970 and G999 of SARS-CoV-2 spike protein (SEQ ID NO:3).

In one aspect, an engineered peptide comprises a coronavirus S1/S2 prefusion spike peptide sequence having one or more amino acid substitutions, mutations, deletions, insertions or combinations thereof. In certain embodiments, the coronavirus S1/S2 prefusion spike peptide sequence comprises two amino acid substitutions to form a disulfide bridge between the two substituted amino acids. In certain embodiments, the two amino acids are substituted at conserved amino acid positions of the coronavirus S1/S2 prefusion spike peptide sequence. In certain embodiments, the two amino acids comprise cysteine, and form a disulfide bridge. In certain embodiments, the coronavirus S1/S2 prefusion spike peptide comprises peptide having a 90% sequence identity to S₉₆₇SNF₉₇₀GAISSVLNDILSRLDKVEAEVQIDRLITG₉₉₉R₁₀₀₀ (SEQ ID NO: 1). In certain embodiments, the coronavirus S1/S2 prefusion spike peptide comprises an amino acid sequence comprising S₉₆₇SNF₉₇₀GAISSVLNDILSRLDKVEAEVQIDRLITG₉₉₉R₁₀₀₀ (SEQ ID NO: 1). In certain embodiments, the coronavirus S1/S2 prefusion spike peptide sequence comprises a cysteine substitution at amino acid position 970. In certain embodiments, the coronavirus S1/S2 prefusion spike peptide sequence comprises a cysteine substitution at amino acid position 999. In certain embodiments, the coronavirus S1/S2 prefusion spike peptide sequence comprises a cysteine substitution at amino acid positions 970 and 999: S₉₆₇SNC₉₇₀GAISSVLNDILSRLDKVEAEVQIDRLITC₉₉₉R₁₀₀₀ (SEQ ID NO: 4). In certain embodiments, the cysteines at amino acid positions 970 and 999 form a disulfide bridge.

In another aspect, an engineered peptide comprises an amino acid sequence having a 90% amino acid sequence identity to S₉₆₇SNF₉₇₀GAISSVLNDILSRLDKVEAEVQIDRLITG₉₉₉R₁₀₀₀ (SEQ ID NO: 1). In certain embodiments, an engineered peptide comprises an amino acid sequence, S₉₆₇SNF₉₇₀GAISSVLNDILSRLDKVEAEVQIDRLITG₉₉₉R₁₀₀₀ (SEQ ID NO: 1), wherein phenylalanine at position 970 (F970) and glycine at position 999 (G999) of SEQ ID NO: 1 are substituted with cysteines (SEQ ID NO:4).

In another aspect the disclosure provides an engineered protein (e.g., a engineered coronavirus spike protein) comprising a coronavirus S1/S2 prefusion spike peptide sequence described herein. In embodiments, the engineered protein is or comprises a stabilized coronavirus S1/S2 prefusion spike protein comprising an amino acid sequence, S₉₆₇SNF₉₇₀GAISSVLNDILSRLDKVEAEVQIDRLITG₉₉₉R₁₀₀₀ (SEQ ID NO: 1), wherein phenylalanine at position 970 (F970) and glycine at position 999 (G999) of SEQ ID NO: 1 are substituted with cysteines.

In another aspect, an engineered protein comprises a coronavirus S1/S2 prefusion spike peptide or protein wherein the coronavirus S1/S2 prefusion spike peptide or protein has an engineered disulfide bond comprising paired cysteine substitutions at positions corresponding to F970 and G999 of SEQ ID NO: 3, including e.g., F1060 and G1089; F1036 and G1065; F1044 and G1073; F952 and G981; F839 and G868; F843 and G872; F1064 and G1093; F1020 and G1049, depending on the position of the F and G in the particular coronavirus spike protein.

In another aspect, an engineered protein comprises a coronavirus S1/S2 prefusion spike protein comprising one or more modifications that stabilize the prefusion S1/S2 prefusion spike protein. In certain embodiments, the modifications include (a) a mutation that inactivates the S1/S2 cleavage site, and (b) an engineered disulfide bond. In certain embodiments, the activation site S2′ (₈₁₄KRSF₈₁₇) in the S2 domain at the FPPR/FP boundary, comprises a mutation resistant to cleavage by membrane-bound host proteases, such as, for example, TMPRSS2 or cathepsin. In certain embodiments, the engineered disulfide bond comprises paired cysteine substitutions at positions corresponding to F970 and G999 of SEQ ID NO: 3, including, e.g., F1060 and G1089; F1036 and G1065; F1044 and G1073; F952 and G981; F839 and G868; F843 and G872; F1064 and G1093; F1020 and G1049, depending on the position of the F and G in the particular coronavirus spike protein.

In another aspect, an engineered protein comprises a stabilized coronavirus S1/S2 prefusion spike protein comprising an engineered disulfide bond comprising paired cysteine substitutions at positions corresponding to F970 and G999 of SEQ ID NO: 3, including, e.g., F1060 and G1089; F1036 and G1065; F1044 and G1073; F952 and G981; F839 and G868; F843 and G872; F1064 and G1093; F1020 and G1049, depending on the position of the F and G in the particular coronavirus spike protein.

In another aspect, a vaccine comprises an immunogenic peptide comprising a 90% sequence identity to S₉₆₇SNF₉₇₀GAISSVLNDILSRLDKVEAEVQIDRLITG₉₉₉R₁₀₀₀ (SEQ ID NO: 1). In certain embodiments, the immunogenic peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1). In certain embodiments, phenylalanine at position 970 (F970) and glycine at position 999 (G999) of SEQ ID NO: 1 are substituted with cysteines. In certain embodiments, the cysteines at amino acid positions 970 and 999 form a disulfide bridge.

In another aspect, a vaccine comprises a stabilized coronavirus S1/S2 prefusion spike protein, wherein the coronavirus S1/S2 prefusion spike protein includes an amino acid sequence, S₉₆₇SNF₉₇₀GAISSVLNDILSRLDKVEAEVQIDRLITG₉₉₉R₁₀₀₀ (SEQ ID NO: 1), wherein phenylalanine at position 970 (F970) and glycine at position 999 (G999) of SEQ ID NO: 1 are substituted with cysteines.

In another aspect, an expression vector encoding a coronavirus S1/S2 prefusion spike peptide comprises a 90% sequence identity to S₉₆₇SNF₉₇₀GAISSVLNDILSRLDKVEAEVQIDRLITG₉₉₉R₁₀₀₀ (SEQ ID NO: 1). In certain embodiments, the coronavirus S1/S2 prefusion spike peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1). In certain embodiments, phenylalanine at position 970 (F970) and glycine at position 999 (G999) of SEQ ID NO: 1 are substituted with cysteines. In certain embodiments, the cysteines at amino acid positions 970 and 999 form a disulfide bridge.

In another aspect, the expression vector encodes a stabilized coronavirus S1/S2 prefusion spike protein, wherein the coronavirus S1/S2 prefusion spike protein includes an amino acid sequence, S₉₆₇SNF₉₇₀GAISSVLNDILSRLDKVEAEVQIDRLITG₉₉₉R₁₀₀₀ (SEQ ID NO: 1), wherein phenylalanine at position 970 (F970) and glycine at position 999 (G999) of SEQ ID NO: 1 are substituted with cysteines.

In another aspect, a host cell comprises an expression vector encoding a coronavirus S1/S2 prefusion spike peptide comprising a 90% sequence identity to S₉₆₇SNF₉₇₀GAISSVLNDILSRLDKVEAEVQIDRLITG₉₉₉R₁₀₀₀ (SEQ ID NO: 1). In certain embodiments, the coronavirus S1/S2 prefusion spike peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1). In certain embodiments, phenylalanine at position 970 (F970) and glycine at position 999 (G999) of SEQ ID NO: 1 are substituted with cysteines. In certain embodiments, the cysteines at amino acid positions 970 and 999 form a disulfide bridge. In certain embodiments, the host cell comprises an autologous cell, an allogeneic cell, a haplotype matched cell, a haplotype mismatched cell, a haplo-identical cell, a xenogeneic cell, stem cell, cell lines, immune system cells or combinations thereof.

A method of preventing infection and treating a subject infected with a coronavirus, comprises administering to the subject a pharmaceutical composition comprising a therapeutically effective amount of a coronavirus S1/S2 prefusion spike peptide or expression vector encoding the coronavirus S1/S2 prefusion spike peptide, wherein the coronavirus S1/S2 prefusion spike peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1). In certain embodiments, phenylalanine at position 970 (F970) and glycine at position 999 (G999) of SEQ ID NO: 1 are substituted with cysteines. In certain embodiments, the method of treating or preventing a coronavirus infection in a subject, further comprises administering to the subject an agent or vaccine. In certain embodiments, the agent comprises an anti-viral agent, an immunomodulatory agent, an antibody, an antibody fragment, a chemotherapeutic agent, or a biological agent.

In another aspect, an engineered peptide comprises an amino acid sequence having a 90% amino acid sequence identity to S₉₆₇SNF₉₇₀GAISSVLNDILSRLDKVEAEVQIDRLITG₉₉₉R₁₀₀₀ (SEQ ID NO: 1), wherein the peptide is conjugated to a secondary agent. In certain embodiments, the engineered peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1).

In another aspect, a composition comprises two or more conjugated peptides wherein the peptides comprise an amino acid sequence having a 90% amino acid sequence identity to S₉₆₇SNF₉₇₀GAISSVLNDILSRLDKVEAEVQIDRLITG₉₉₉R₁₀₀₀ (SEQ ID NO: 1). In certain embodiments, the two or more conjugated peptides comprise at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1). In certain embodiments, the two or more peptides are conjugated via a linker molecule. In certain embodiments, the two or more peptides are fused to each other. In certain embodiments, the composition further comprises an adjuvant.

In another aspect, the disclosure provides a nucleic acid that encodes a coronavirus S1/S2 prefusion spike peptide or protein described herein. In embodiments, the nucleic acid is or comprised RNA. In embodiments, the nucleic acid is or comprised DNA. In embodiments, the nucleic acid molecule comprises a nucleotide sequence encoding an amino acid sequence having a 90% amino acid sequence identity to S₉₆₇SNF₉₇₀GAISSVLNDILSRLDKVEAEVQIDRLITG₉₉₉R₁₀₀₀ (SEQ ID NO: 1). In certain embodiments, the nucleic acid molecule encodes comprises an amino acid sequence comprising at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1).

In another aspect, a nucleic acid molecule encodes a stabilized coronavirus S1/S2 prefusion spike protein, wherein the coronavirus S1/S2 prefusion spike protein includes an amino acid sequence, S₉₆₇SNF₉₇₀GAISSVLNDILSRLDKVEAEVQIDRLITG₉₉₉R₁₀₀₀ (SEQ ID NO: 1), wherein phenylalanine at position 970 (F970) and glycine at position 999 (G999) of SEQ ID NO: 1 are substituted with cysteines.

In another aspect, a method of identifying candidate therapeutic agents for preventing or treating a coronavirus infection comprises contacting a substrate comprising: (i) a nucleic acid molecule encoding a peptide having a 90% amino acid sequence identity to S₉₆₇SNF₉₇₀GAISSVLNDILSRLDKVEAEVQIDRLITG₉₉₉R₁₀₀₀ (SEQ ID NO: 1); or, (ii) a peptide having a 90% amino acid sequence identity to S₉₆₇SNF₉₇₀GAISSVLNDILSRLDKVEAEVQIDRLITG₉₉₉R₁₀₀₀ (SEQ ID NO: 1); or, (iii) a nucleic acid molecule encoding a peptide comprising at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1), with a candidate therapeutic agent; or, (iv) a peptide comprising at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1), with a candidate therapeutic agent; or, (v) a cell comprising any one of nucleic acids or peptides of (i)-(iv); and conducting an assay for an output value. In certain embodiments, the assay comprises: immunoassays, Southern blots, Western blots, polymerase chain reaction (PCR), Northern blots, sequencing, reverse-transcriptase PCR, microarray technology, immunohistochemistry, enzyme-linked immunosorbent assay, flow cytometry mass spectrometry, Förster resonance energy transfer, time-resolved fluorescence energy transfer, amplified luminescent proximity homogeneous assay, fluorescence polarization, cell based assays or combinations thereof. In certain embodiments, the assay is a high throughput screening (HTS) assay.

In another aspect, the disclosure provides a coronavirus S1/S2 prefusion spike peptide or protein comprising a F to C (Phe to Cys) substitution and a G to C (Gly to Cys) substitution at positions corresponding to F970 and G999 of SARS-CoV-2 spike protein (SEQ ID NO: 3). In embodiments, the S1/S2 prefusion spike peptide or protein comprises a disulfide bridge between the two cysteines. In another aspect, a coronavirus S1/S2 prefusion spike peptide or protein comprises a disulfide bridge between a cysteine substituted for a phenylalanine (Phe) located between amino acid position 800 to about amino acid position 1100 and a cysteine (Cys) substituted for a glycine (Gly) located 29 amino acids from the Phe amino acid (in other words, the Phe to Cys substitution and the Gly to Cys substitution are separated by 28 intervening amino acid residues), wherein the cysteines form a disulfide bridge. In certain embodiments, coronavirus S1/S2 prefusion spike protein comprises a Phe at amino acid position 970 and a Gly at amino acid position 999, which are each substituted with a cysteine. In certain embodiments, the coronavirus S1/S2 prefusion spike protein comprises a Cys substituted for Phe at amino acid position 1060 and a Cys substituted for Gly at amino acid position 1089. In certain embodiments, the coronavirus S1/S2 prefusion spike protein comprises a Cys substituted for Phe at amino acid position 1036 and a Cys substituted for Gly at amino acid position 1065. In certain embodiments, the coronavirus S1/S2 prefusion spike protein comprises a Cys substituted for Phe at amino acid position 1044 and a Cys substituted for Gly at amino acid position 1073. In certain embodiments, the coronavirus S1/S2 prefusion spike protein comprises a Cys substituted for Phe at amino acid position 952 and a Cys substituted for Gly at amino acid position 981. In certain embodiments, the coronavirus S1/S2 prefusion spike protein comprises a Cys substituted for Phe at amino acid position 839 and a Cys substituted for Gly at amino acid position 868. In certain embodiments, the coronavirus S1/S2 prefusion spike protein comprises a Cys substituted for Phe at amino acid position 843 and a Cys substituted for Gly at amino acid position 872. In certain embodiments, the coronavirus S1/S2 prefusion spike protein comprises a Cys substituted for Phe at amino acid position 1064 and a Cys substituted for Gly at amino acid position 1093. In certain embodiments, the coronavirus S1/S2 prefusion spike protein comprises a Cys substituted for Phe at amino acid position 1020 and a Cys substituted for Gly at amino acid position 1049.

### Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary.

Standard nomenclature is used for the natural amino acids and their abbreviations. For example, L-alanine is represented with the three-letter abbreviation Ala, or one-letter abbreviation “A”. Where indicated, the “D” stereoisomer of alanine is represented as D-Ala.

Standard nomenclature is used for the bases of DNA, with cytosine, guanosine, adenine, and thymine indicated as “C”, “G”, “A”, and “T”, and codons that encode DNA follow the standard genetic code, for example the amino acid Leu is encoded by TTA, TTG, CTT, CTC, CTA or CTG, and Asp is encoded by GAT or GAC.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude, within 5-fold, and also within 2-fold, of a value. Where particular values are described in the application and claims, unless otherwise stated the term “about” meaning within an acceptable error range for the particular value should be assumed. All numeric values are herein assumed to be modified by the term “about”, whether or not explicitly indicated. The recitation of numerical ranges by endpoints includes all numbers within that range (e.g., 1 to 5 includes 1, 1.5, 2, 2.75, 3, 3.80, 4, and 5).

As used herein, the term “alteration” or “modification” or “mutation” refers to any change in the sequence of a molecule. Modifications may include, but are not limited to, insertions, deletions, substitutions and variants. As used herein, the term “insertion” refers to an addition of one or more nucleotides in a DNA sequence. Insertions can range from small insertions of a few nucleotides to insertions of large segments such as a cDNA or a gene. The term “deletion” refers to a loss or removal of one or more nucleotides in a DNA sequence. In some cases, a deletion can include, for example, a loss of a few nucleotides, an exon, an intron, a gene segment.

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the invention at any stage during clinical trials, during pre-trial testing, or following FDA-approval.

By “ameliorate” is meant decrease, suppress, attenuate, diminish, arrest, or stabilize the development or progression of a disease.

The term “amino acid” as used herein refers to naturally occurring and synthetic α, β, γ, and δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. The amino acids can be non-naturally occurring amino acids. Examples of non-naturally occurring amino acids include, but are not limited to, D-amino acids (i.e., an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine. When the term amino acid is used, it is considered to be a specific and independent disclosure of each of the esters of α, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

The term “amino acid sequence” is the order in which amino acid residues, connected by peptide bonds, lie in the chain in peptides and proteins.

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139(1999); Freier S. M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000)); 2′-O, 3′-C-linked [3.2.0] bicycloarabinonucleosides (see e.g. N. K Christiensen., et al., J. Am. Chem. Soc., 120: 5458-5463 (1998). Such analogs include synthetic nucleosides designed to enhance binding properties, e.g., duplex or triplex stability, specificity, or the like.

As used herein, the term “anti-viral agent” refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, gene-editing agents, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

As used herein, the terms “conjugated,” “linked,” “attached,” “fused” and “tethered,” when used with respect to two or more moieties, means that the moieties or domains are physically associated or connected with one another, either directly or via one or more additional moieties that serve as a linking agent, to form a structure that is sufficiently stable so that the moieties remain physically associated under the conditions in which the structure is used, e.g., physiological conditions. The linkage can be based on genetic fusion according to the methods known in the art or can be performed by, e.g., chemical cross-linking. In certain embodiments, the stabilized coronavirus S1/S2 prefusion spike polypeptides, peptides can be associated with one or more other stabilized coronavirus S1/S2 prefusion spike polypeptides, peptides to form dimers, trimers etc. In certain embodiments, the stabilized coronavirus S1/S2 prefusion spike polypeptides, peptides can be associated with one or more other agents. In certain embodiments, the dimers, trimers of the stabilized coronavirus S1/S2 prefusion spike polypeptides, peptides may be linked by a flexible linker, such as a polypeptide linker. In certain embodiments, the stabilized coronavirus S1/S2 prefusion spike polypeptides, peptides and agents may be linked by a flexible linker, such as a polypeptide linker. The polypeptide linker can comprise plural, hydrophilic or peptide-bonded amino acids of varying lengths.

As used herein, the term “coronavirus” is well known to the person skilled in the art. In general coronaviruses are viruses of the subfamily Coronavirinae in the family Coronavirinae, in the order Noroviruses. Coronaviruses are enveloped viruses and have a positive-sense single-stranded RNA genome with a nucleocapsid of helical symmetry. The term “coronavirus” encompasses all strains, genotypes, protectotypes, and serotypes of this virus.

In the description and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C”, “one or more of A, B, and C” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the transitional term “comprising,” which is synonymous with “including,” “containing,” or “characterized by,” is inclusive or open-ended and does not exclude additional, unrecited elements or method steps. When used herein the term “comprising” can be substituted with the term “containing” or “including” or sometimes when used herein with the term “having.” By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

A “conservative amino acid substitution” is one in which the amino acid residue is replaced with an amino acid residue having a similar sidechain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

A “control” sample or value refers to a sample that serves as a reference, usually a known reference, for comparison to a test sample. For example, a test sample can be taken from a test subject, e.g., a subject with a coronavirus infection and compared to samples from known conditions, e.g., a subject (or subjects) that does not have a coronavirus infection or, in some cases a subject that was previously infected with a coronavirus (a negative or normal control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The terms “corresponding to” or “numbered with reference to” or when used in the context of the numbering of a given amino acid or polynucleotide sequence, refers to the numbering of the residues of a specified reference sequence when the given amino acid or polynucleotide sequence is compared to the reference sequence.

As used herein, “derivative” polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

As used herein, a “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

As used herein, the terms “determining”, “measuring”, “evaluating”, “detecting”, “assessing” and “assaying” are used interchangeably herein to refer to any form of measurement or output value (e.g., signal intensity) and include determining if an element is present or not. These terms include both quantitative and/or qualitative determinations. Assessing may be relative or absolute. “Assessing the presence of” includes determining the amount of something present, as well as determining whether it is present or absent.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “disease” is a state of health of an animal wherein the animal cannot maintain homeostasis, and wherein if the disease is not ameliorated then the animal's health continues to deteriorate.

As used herein, the term “immune system cells”, “immune cells” or “cells of the immune system” refer to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4⁺ T cells or CD8⁺ T cells). In some aspects, an immune cell that can be targeted with a targeting moiety (e.g., anti-CD3) comprises a naive CD4⁺ T cell. In some aspects, an immune cell comprises a memory CD4⁺ T cell. In some aspects, an immune cell comprises an effector CD4⁺ T cell. In some aspects, an immune cell comprises a naïve CD8⁺ T cell. In some aspects, an immune cell comprises a memory CD8⁺ T cell. In some aspects, an immune cell comprises an effector CD8⁺ T cell. In some aspects, an immune cell comprises a gamma delta T cell. In some aspects, an immune cell is an antigen presenting cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof.

The term “linker”, also referred to as a “spacer” or “spacer domain”, refers to an amino acid or sequence of amino acids that that is optionally located between two amino acid sequences in a fusion protein of the invention.

As used herein, the term “kit” refers to any delivery system for delivering materials. Inclusive of the term “kits” are kits for both research and clinical applications. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides or liposomes. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, a “natural amino acid” refers to the twenty genetically encoded alpha-amino acids. See, e.g., Biochemistry by L. Stryer, 3^{rd} ed. 1988, Freeman and Company, New York for structures of the twenty natural amino acids.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise and should be understood to mean “either or both” of the elements so conjoined, e.g., elements that are conjunctively present in some cases and disjunctively present in other cases.

“Parenteral” administration of an immunogenic composition includes, e.g., subcutaneous (s.c.), intravenous (i.v.), intramuscular (i.m.), intravitreal (i.v.i.), intra-cisterna magna (i.c.m.), or intrasternal injection, or infusion techniques.

As used herein, an amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1,-2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neoantigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

The term, “S1/S2 prefusion” spike protein refers to the two functional subunits, designated S1 and S2 of the coronavirus spike (S protein). Coronavirus virions are decorated with a spike (S) glycoprotein that binds to host-cell receptors and mediates cell entry via fusion of the host and viral membranes. S proteins are trimeric class I fusion proteins that are expressed as a single polypeptide that is subsequently cleaved into S1 and S2 subunits by cellular proteases. The S1 subunit contains the receptor-binding domain (RBD), which, in the case of SARS-CoV-2, recognizes the angiotensin-converting enzyme 2 (ACE2) receptor on the host-cell surface. The S2 subunit mediates membrane fusion and contains an additional protease cleavage site, referred to as S2′, that is adjacent to a hydrophobic fusion peptide. Binding of the RBD to ACE2 triggers S1 dissociation, allowing for a large rearrangement of S2 as it transitions from a metastable prefusion conformation to a highly stable postfusion conformation (Hsieh C L, et al. Structure-based Design of Prefusion-stabilized SARS-CoV-2 Spikes. bioRviv 2020 May 30:2020.05.30.125484. doi: 10.1101/2020.05.30.125484; PMID: 32577660; PMCID: PMC7302215). See, also FIGS. 1A-1E. Accordingly, “a stabilized coronavirus S1/S2 prefusion spike protein” refers to a spike protein which includes mutations to the S protein, as disclosed herein, to maintain the S1 and S2 in the prefusion conformation. In particular embodiments, the coronavirus S1/S2 prefusion spike proteins include a disulfide bond that “staple” together the central helix (CH) and a region of the spike known as HR1. By preventing HR1 from detaching from CH, the prefusion spike structure is stabilized without rigidification of the central helix or changes to its interaction with the receptor binding domain. This disulfide-stapled spike is more stable in the prefusion form, allowing for a stable vaccine without the need for the stabilizing mutations that are currently in use. A stabilized prefusion conformation of class I fusion proteins is desirable for vaccine development because this conformation is found on infectious virions and displays most or all of the neutralizing epitopes that can be targeted by antibodies to prevent the entry process.

The term “subject” refers to any animal classified as a mammal, such as humans and non-human mammals. Examples of non-human animals include dogs, cats, cows, horses, sheep, pigs, goats, rabbits, and the like. The terms “patient” or “subject” are used interchangeably herein, unless otherwise indicated. Preferably, the subject is a human.

As used herein, a “therapeutically effective” amount of a compound or agent (i.e., an effective dosage) means an amount sufficient to produce a therapeutically (e.g., clinically) desirable result. The compositions can be administered from one or more times per day to one or more times per week, including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the disclosure can include a single treatment or a series of treatments.

The terms “treat,” “treating” or “ameliorating” include administering a compound or agent to a subject to prevent or delay the onset of symptoms, complications or biochemical indicators of a disease (e.g., a coronavirus infection), alleviate symptoms or arrest or inhibit further development of the disease, condition or disorder. Subjects in need of treatment include those already with the disease or disorder as well as those at risk of developing the disorder. Treatment may be prophylactic (to prevent or delay the onset of the disease, or to prevent the manifestation of clinical or subclinical symptoms thereof) or may be therapeutic inhibition or symptomatic relief following the manifestation of the disease.

As used herein, an “unnatural amino acid,” “non-natural”, “modified amino acid” or “chemically modified amino acid” refers to any amino acid, modified amino acid, or amino acid analogue other than the twenty genetically encoded alpha-amino acids. Unnatural amino acids have side chain groups that distinguish them from the natural amino acids, although unnatural amino acids can be naturally occurring compounds other than the twenty proteinogenic alpha-amino acids. In addition to side chain groups that distinguish them from the natural amino acids, unnatural amino acids may have an extended backbone such as beta-amino acids.

Non-limiting examples of non-natural amino acids include selenocysteine, pyrrolysine, homocysteine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-G1cNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline. In an embodiment of the coronavirus S1/S2 prefusion spike polypeptides described herein, one or more amino acids of the coronavirus S1/S2 prefusion spike peptide are substituted with one or more unnatural amino acids and/or one or more natural amino acids.

The term “vaccine” refers to a pharmaceutical composition that elicits a prophylactic or therapeutic immune response in a subject. In some cases, the immune response is a protective immune response. Typically, vaccines elicit antigen-specific immune responses against pathogens, such as antigens of viral pathogens or cellular components associated with pathological conditions. A vaccine can include a polynucleotide (e.g., a nucleic acid encoding a known antigen), a peptide or polypeptide (e.g., a disclosed antigen), a virus, a cell, or one or more cellular components. In some embodiments, the vaccine or vaccine antigen or vaccine composition is expressed from a vector.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the disclosure are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “vector” refers to a nucleic acid molecule capable of transporting another nucleic acid to which it has been linked. One type of vector is a “plasmid”, which refers to a circular double-stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In some examples, vectors can be capable of directing the expression of nucleic acids to which they are operatively linked. Such vectors are referred to herein as “recombinant expression vectors”, or more simply “expression vectors”, which serve equivalent functions.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human. The term “gene” is also intended to include variants.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1E show structure models along the presumed pathway for spike-mediated membrane fusion in SARS-CoV-2. Each spike protomer is shown in a different primary color, ACE2 is shown in pink, and glycans are indicated in gray. FIG. 1A: The pre-fusion spike contains both S1 and S2 subunits. FIG. 1B: The spike binds to ACE2 using an open RBD. FIG. 1C: The S1 subunit sheds, leaving a metastable S2. FIG. 1D: The HR1 are released, extending the CH coiled coil and inserting the FP into the host cell membrane. FIG. 1E: HR1 and HR2 come together to form a 6-helix bundle, merging the viral and host membranes.

FIG. 2 is a schematic showing the structure of the pre-fusion spike ectodomain with 3 open RBDs (7CAK). S1 subunits are shown in a space-filling model and S2 are drawn with ribbons. For clarity the S1 subunit is not shown for one protomer (yellow). Each protomer is a different primary color, with one S2 loop highlighted in orange. Inset: close-up view of the top of the S2 subunit for one protomer in wild type 6XR8.

FIG. 3 shows a comparison of the S2 core in the pre-fusion (upper, 6XR8) and post-fusion (lower, 6XRA) spike. Amino acids on the pre-fusion CH (V987 to A1015) are shown as space-filling. The distance between N751 on neighboring UH for one pair of protomers is indicated with a pink line. After removal of the SRL, rotation and repacking of S2 results in a notable size reduction in the S2 subunit.

FIG. 4 shows a comparison of features between the upper S2 subunit of the wild-type spike in the pre-fusion 6XR8 (left, copper), intermediate model (middle, primary colors), and post-fusion 6XRA (right, gray) structures. The S1 subunits in the left and middle structures are present but omitted for clarity; the post-fusion spike contains only S2. FIG. 20 shows these regions with labels on selected amino acids.

FIG. 5 shows the structure of the 3-up spike with unfolded SRL and partially extended CH, comparable to the pre-fusion structure in FIG. 2. Protomers are shown as primary colors, with the S1 subunits in space-filling and the S2 subunits as ribbons. The yellow S1 subunit is omitted to provide a clear view of the S2 subunit.

FIG. 6A shows a structure of the trimeric SARS-CoV-2 spike glycoprotein ectodomain, with the S1 subunit shown in green, the S2 subunit shown in blue, and the glycans in gray. FIG. 6B: Ribbon drawing of a single protomer, color coded by domains indicated in the lower figure. Domain labels are adapted from Y. Cai et al. Science 369, 1586-1592 (2020).

FIG. 7 shows that the side chain of R1000 on the CH is surrounded by hydrophobic amino acids. The phenyl ring of F970 on the SRL is packed against the α-carbon of G999 on the CH. The structure shown is the wild-type spike in the pre-fusion conformation (6XR8). The SRL (shown transparent to aid visualization) forms a cap over the hydrophobic pocket.

FIG. 8 is a series of schematics showing a top view of the pre-fusion spike 6XR8, showing locations of NTD and RBD domains of the S1 subunit atop the spike. Left: Each protomer is shown in a different color space-filling model (red, blue and yellow in a clockwise rotation). Right: The S1 subunit is shown in ribbon drawing, while the lower S2 subunit is shown as space-filling. The S2 protomers are shown in a darker shade than the same protomer in the S1 subunit. Each RBD covers the S2 subunit of both of the other protomers; correspondingly, each S2 subunit is covered by the RBD domains of both of the other protomers.

FIG. 9 shows that the RBD domains of 2 different protomers cover the helix-turn-helix involving CH and HR1 at the top of one S2 protomer. Ribbons are colored by protomer. Labeled amino acids are discussed in the main text. The structure shown is the wild-type spike in the pre-fusion conformation (6XR8).

FIG. 10 is a series of schematics showing the contacts between the CTD1 and the HR1 upper short helix, in 3 cryo-EM structures of the pre-fusion spike. Ribbons are colored by protomer. Left: 6XR8, closed. Middle: 6VSB, open unbound. Right: 7CAK, 3 RBDs open and bound. As the RBD opens, the distance between CTD1 and HR1 increases, resulting in loss of contacts between the S1 and S2 subunits (D571-5975 and R567-D979).

FIGS. 11A-11L are a series of histograms and plots showing data extracted from 80 SARS-CoV-2 spike pre-fusion structures obtained from the PDB (PDB codes provided in Table 1). FIG. 11A: Histogram of distances from RBD to the top of HR1 or CH (the leftmost peak corresponds to closed RBDs). FIG. 11B: Histogram of distances from R815 at the S2′ site to nearby A871. FIG. 11C: Histogram of distances for the close approach of the ends of the SRL, from 5967 to 5975. FIG. 11D: Backbone Ramachandran plot for F970, showing that all pre-fusion structures (black) adopt a high-energy region of the phi/psi space, in contrast to the post-fusion 6XRA (red). FIG. 11E: Histogram of distances from the F970 phenyl ring to G999. FIG. 11F: Histogram of distances for the hydrogen bond from R1000 side chain to the S975 backbone O. FIG. 11G: Histogram of distances for the hydrogen bond from R1000 side chain to the 1742 backbone O. FIG. 11H: X-axis, distance from RBD to the top of HR1 (S383 N to R983 O); Y-axis, C-capping distance from K386 NZ (RBD) to I981 O (HR1) (see FIG. 9). FIG. 11I: X-axis, distance from RBD to the top of HR1 (S383 N to R983 O); Y-axis, hydrogen bonding distance from D571 (CTD1) to S975 (HR1) (see FIG. 10). FIG. 11J: X-axis, distance from RBD to the top of HR1 (S383 N to R983 O); Y-axis, salt bridge distance from R567 (CTD1) to D979 (HR1) (see FIG. 10). FIG. 11K: Histogram of distances for possible salt bridge between D994 (CH) and R995′ (CH′) (FIG. 12). FIG. 11L: histogram of distances corresponding to edges of the UH-UH triangle (FIG. 3): distance from N751 CA to N751′ CA.

FIG. 12 shows views of selected amino acids at the top of the wild-type spike S2 subunit in the pre-fusion (left, 6XR8, copper) and post-fusion (right, 6XRA, silver) structures. In the post-fusion spike, six new salt bridges have formed, separated by only 3 turns of α-helix. These involve D994/R995′ and R983/D985′ on the CH of neighboring protomers. A new hydrophobic cluster stabilizes the post-fusion coiled-coil motif above R983.

FIG. 13 shows a cross-section of the wild-type spike near the SRL, viewed from the top. (Top left) pre-fusion (6XR8, copper) and (bottom left) post-fusion (6XRA, silver), and (right) both structures best-fit to CH. Labels are used on selected amino acids for one protomer. In the pre-fusion structure, the SRL inserts between the CH and UH of neighboring protomers. In the post-fusion structure, SRL is relocated, the CH and neighbor UH are in contact, and salt bridges D994-R995′ form between the CH of neighboring protomers. Overlap indicates that the SRL blocks the closer approach of CH and neighbor UH in the pre-fusion structure.

FIG. 14: The upper S2 rotates counter-clockwise relative to the lower S2 during spike activation (indicated by red arrows). Top view (left) and side view (right) of the wild-type spike in pre-fusion (copper, 6XR8) and post-fusion (silver, 6XRA) structures. The structures are best-fit to the lower part of the spike (L1049MSFPQSAP1057) in the connector domain (CD). The fulcrum for rotation of the upper S2 is indicated by the red box drawn at M731 for UH, and E1017 for CH. For clarity, some regions are omitted from view.

FIG. 15 shows an RMSD analysis of the closed and 3-up standard MD simulations of the full spike ectodomain. Each column represents a different protomer. The different colors on each plot represent four independent trials of ˜400 ns each that were carried out for closed and 3-up spike. The upper S2 region includes positions 737-761 (UH) and 962-1005 (HR1-SRL-CH). The SRL region includes positions 967-976. The reference structure for all plots was the 6XR8 closed structure. The S2 and SRL are stable in all runs.

FIG. 16 shows a water density grid (purple) inside the hydrophobic pocket that encloses R1000, calculated during MD simulation of the closed full spike ectodomain. Similar water occupancy at this location is observed in all MD simulations.

FIG. 17 shows two model systems that were used for simulations of the upper region of the S2 subunit. Left: the pre-fusion spike trimer 6XR8, with the 51 subunit in gray and S2 colored by protomer. The box indicates the area corresponding to the model systems. Middle: the medium model system, retaining only the CH, HR1, SRL and UH, truncated at the bottom. Right: the small model system obtained by removing the SRL and lower helix of HR1 from the medium model (indicated by the dashed box).

FIG. 18 shows the results of standard all-atom MD in explicit water on the small model system compared to the post-fusion spike 6XRA. (Upper) Small model structure (primary colors, UH in darker shade) and post-fusion spike (gray, right), all best-fit to amino acids in the CH near the base of the model system (Q1005TYVTQQLIRAA1016). The initial conformation (left, initiated from the pre-fusion structure 6XR8) differs mainly in the helix-turn-helix motif at the top of the CH. After 1.5 pec standard MD (middle), the upper short helix segments of HR1 have spontaneously rotated upward and lengthened the CH of each protomer. (Lower) Time-dependent backbone RMSD values of each protomer for the upper HR1 helix-4 segment (D979ILSRL984) in simulations compared to the post-fusion structure (after best-fit to CH K986VEAEVQIDRLITG999). Left: wild-type sequence; right: including 2P substitution, in longer simulations. The HR1 rotates to extend the CH in the wild type, but not in the 2P system.

FIG. 19 show that the unfolding of the SRL allows extension of the central helix. Medium model system before (left) and after (right) extension of the CH by using SMD on the HR1 helix-4 to match the structure obtained in the small model MD (FIG. 18). Protomers are shown in light primary colors, and the SRL is shown in dark blue.

FIG. 20 shows a comparison of the final structure from MD on the S2 medium model to the post-fusion spike. (Left) MD structure after CH extension, with each protomer in a different primary color (CH/HR1 lighter, UH darker). N978 forms an N-capping interaction on the CH, similar to that involving D985 in the pre-fusion structure (FIG. 2). Other contacts that form are comparable to the post-fusion structure (right image), including six additional salt bridges between neighboring CH pairs, and a hydrophobic cluster involving 1980 and L984 that stabilizes the central coiled coil.

FIG. 21 shows a comparison of the medium model system after MD (primary colors) with the post-fusion structure 6XRA (gray). The unfolded SRL follows a similar path alongside CH to that taken by a segment of HR2 in the post-fusion structure. The side chain of F970 is positioned similarly to F1156 on HR2 in the post-fusion structure. For clarity, some regions are omitted.

FIG. 22 shows snapshots along the pathway for RBD extension in the 3-up full spike ectodomain system (with the initial pre-fusion structure shown on the left). A single protomer of S2 is shown using purple ribbons, and the S1 subunits are shown in space-filling with a different color for each protomer. For clarity, only a single protomer of S2 is shown, and the RBD of the yellow S1 subunit is omitted to provide a clear view of the S2.

FIGS. 23A-23F demonstrate the time dependence of changes in full spike ectodomain system during the three steps of (left column) unrestrained MD for 3-up spike; (middle column) SMD for extension of CH using HR1 helix-4; (right column) unrestrained MD of 3-up spike after SMD. Each protomer is shown in a different color. FIG. 23A: Distance between CA atoms of S₉₆₇ and S975 of the SRL that approach closely in the pre-fusion state (structure in FIG. 2); FIG. 23B: salt bridge distance between D994 (CH) and R995′ (CH) on the neighboring protomer (structure in FIG. 20); FIG. 23C: salt bridge distance between R983 (HR1) and D985′ (CH) of the neighboring protomer (structure in FIG. 20); FIG. 23D: distance between CG atoms of 1980 (HR1) and 1980′ of the neighboring protomer (structure in FIG. 20), FIG. 23E: Backbone RMSD of the upper HR1 segment (D979ILSRL984) in simulations compared to the post-fusion structure (as shown in FIG. 18 for the model system), FIG. 23F: twist of the UH in each protomer (structure in FIG. 14). Upper and lower lines indicate values from the pre-fusion and post-fusion cryo-EM structures, respectively. After partial extension of CH, the newly formed contacts are stable during 250 ns of unrestrained MD, and the UH slowly relax toward the value adopted in the post-fusion structure.

FIG. 24 shows the combined solvation free energy of the three R1000 amino acids, during MD trajectories of different spike conformations. Calculations were performed on MD snapshots using the Poisson-Boltzmann method. Blue: closed; orange: 3-up, green: 3-up, partially extended CH. The extension of CH and unfolding of the SRL leads to significantly better solvation energy for R1000.

FIG. 25 shows the overlap of one protomer of S2 from: (green) a full-spike ectodomain MD simulation with the F970C/G999C substitution and disulfide bond, and (gray) the pre-fusion structure 6XR8 (F970/G999). Disulfide bonds and F970/G999 are shown in licorice. The introduction of the disulfide is accommodated in simulations with minimal perturbation. For clarity, other protomers and the S1 subunit are not shown.

FIG. 26A-26B. FIG. 26A is an image showing overlaid structures for all 9 coronavirus spike structures that have been determined experimentally (currently, only 9 different coronaviruses have known spike structures). The PDB codes and other information are provided in the table below (FIG. 26B). Each structure shows the presence of a conserved loop region that interrupts helix-3 and helix-4 of the HR1 (heptad repeat 1) segment of the spike. Each has a Phe and Gly at the same relative location in the structure, e.g., at positions corresponding to F970 and G999 of the SARS-COV-2 (Phe in stick model, Gly alpha carbon in a sphere). Although the position numbers (relative positions in the protein) change, they are always spaced by 29 amino acids. The Phe is part of the loop while the Gly is part of the central helix. The disulfide staple is intended to irreversibly link this loop to the central helix, blocking activation of the spike (based on our simulation models of the spike). Based on these structures and sequences, the disulfide bridge would have the same stabilization effect on all coronavirus spikes.

FIG. 27 is a schematic showing sequences for a variety of coronaviruses. The sequence segment covers the region connecting HR1 to CH. The positions corresponding to F970 and G999 in SARS-CoV-2 are highly conserved and marked with the black arrows.

FIG. 28 is a histogram of distances between F970 (beta carbon) and G999 (alpha carbon) across 566 experimental structures of the SARS-CoV-2 spike. This measurement was made using a spike analysis tool that we recently published (“SpikeScape: A Tool for Analyzing Structural Diversity in Experimental Structures of the SARS-CoV-2 Spike Glycoprotein”, doi.org/10.1021/acs.jcim.2c01366, incorporated herein by reference in its entirety).

### DETAILED DESCRIPTION

Coronaviridae is a family of enveloped, positive-strand RNA viruses that infect a wide variety of animals. The Coronaviridae family belongs to the suborder Cornidovirineae, which, together with Tornidovirineae belong to the order Nidovirales (enveloped, positive-strand RNA viruses). Recent phylogenetic studies based on RNA-directed RNA polymerases indicate that Nidovirales, together with Picornavirales, Caliciviridae, Astroviridae, and their relatives form a distinct supergroup of RNA viruses (Picornavirus supergroup) (V. V. Dolja, E. V. Koonin. Metagenomics reshapes the concepts of RNA virus evolution by revealing extensive horizontal virus transfer Virus Res., 244 (2020), pp. 36-52; Wolf, D. et al., Origins and evolution of the global RNA virome. mBio, 9 (2018), pp. 1-31, 10.1128/mBio.02329-18). Nidovirales can infect a wide range of animal hosts, including insects, mollusks, crustaceans, and vertebrates, suggesting horizontal virus transfer across metazoan species. Coronaviridae are divided into two subfamilies Letovirinae and Orthocoronavirinae. Orthocoronavirinae in turn are divided into four genera, Alpha-, Beta-, Gamma-, and Delta-coronaviruses. Currently, there are seven Orthocoronavirinae species or sub-species, which have been found to infect humans, two members of the Alphacoronavirus genus: Human coronavirus 229E and Human coronavirus NL63, and five members of the Betacoronavirus genus: Human coronavirus OC43, Human coronavirus HKU1, Middle East respiratory syndrome-related coronavirus (MERS-CoV), Severe acute respiratory syndrome coronavirus (SARS-CoV), and Severe acute respiratory syndrome coronavirus 2 (2019-nCoV, SARS-CoV-2 (K. G. Andersen, et al. The proximal origin of SARS-CoV-2. Nat. Med., 26, 450-452 (2020). doi.org/10.1038/s41591-020-0820-9).

An example of a coronavirus is the Severe acute respiratory syndrome coronavirus 2 isolate Wuhan-Hu-1. The nucleic acid sequence NCBI Reference Sequence of Severe acute respiratory syndrome coronavirus 2 isolate Wuhan-Hu-1, complete genome: accession NC_045512.2, incorporated herein in its entirety (SEQ ID NO: 2):

| | |
|---|---|
| 1 | atgtttgttt ttcttgtttt attgccacta gtctctagtc agtgtgttaa tcttacaacc |
| 61 | agaactcaat taccccctgc atacactaat tctttcacac gtggtgttta ttaccctgac |
| 121 | aaagttttca gatcctcagt tttacattca actcaggact tcttcttacc tttcttttcc |
| 181 | aatgttactt ggttccatgc tatacatgtc tctgggacca atggtactaa gaggtttgat |
| 241 | aaccctgtcc taccatttaa tgatggtgtt tattttgctt ccactgagaa gtctaacata |
| 301 | ataagaggct ggatttttgg tactacttta gattcgaaga cccagtccct acttattgtt |
| 361 | aataacgcta ctaatgttgt tattaaagtc tgtgaatttc aattttgtaa tgatccattt |
| 421 | ttgggtgttt attaccacaa aaacaacaaa agttggatgg aaagtgagtt cagagtttat |
| 481 | tctagtgcga ataattgcac ttttgaatat gtctctcagc cttttcttat ggaccttgaa |
| 541 | ggaaaacagg gtaatttcaa aaatcttagg gaatttgtgt ttaagaatat tgatggttat |
| 601 | tttaaaatat attctaagca cacgcctatt aatttagtgc gtgatctccc tcagggtttt |
| 661 | tcggctttag aaccattggt agatttgcca ataggtatta acatcactag gtttcaaact |
| 721 | ttacttgctt tacatagaag ttatttgact cctggtgatt cttcttcagg ttggacagct |
| 781 | ggtgctgcag cttattatgt gggttatctt caacctagga cttttctatt aaaatataat |
| 841 | gaaaatggaa ccattacaga tgctgtagac tgtgcacttg accctctctc agaaacaaag |
| 901 | tgtacgttga aatccttcac tgtagaaaaa ggaatctatc aaacttctaa ctttagagtc |
| 961 | caaccaacag aatctattgt tagatttcct aatattacaa acttgtgccc ttttggtgaa |
| 1021 | gtttttaacg ccaccagatt tgcatctgtt tatgcttgga acaggaagag aatcagcaac |
| 1081 | tgtgttgctg attattctgt cctatataat tccgcatcat tttccacttt taagtgttat |
| 1141 | ggagtgtctc ctactaaatt aaatgatctc tgctttacta atgtctatgc agattcattt |
| 1201 | gtaattagag gtgatgaagt cagacaaatc gctccagggc aaactggaaa gattgctgat |
| 1261 | tataattata aattaccaga tgattttaca ggctgcgtta tagcttggaa ttctaacaat |
| 1321 | cttgattcta aggttggtgg taattataat tacctgtata gattgtttag gaagtctaat |
| 1381 | ctcaaacctt ttgagagaga tatttcaact gaaatctatc aggccggtag cacaccttgt |
| 1441 | aatggtgttg aaggttttaa ttgttacttt cctttacaat catatggttt ccaacccact |
| 1501 | aatggtgttg gttaccaacc atacagagta gtagtacttt cttttgaact tctacatgca |
| 1561 | ccagcaactg tttgtggacc taaaaagtct actaatttgg ttaaaaacaa atgtgtcaat |
| 1621 | ttcaacttca atggtttaac aggcacaggt gttcttactg agtctaacaa aaagtttctg |
| 1681 | cctttccaac aatttggcag agacattgct gacactactg atgctgtccg tgatccacag |
| 1741 | acacttgaga ttcttgacat tacaccatgt tcttttggtg gtgtcagtgt tataacacca |
| 1801 | ggaacaaata cttctaacca ggttgctgtt ctttatcagg atgttaactg cacagaagtc |
| 1861 | cctgttgcta ttcatgcaga tcaacttact cctacttggc gtgtttattc tacaggttct |
| 1921 | aatgtttttc aaacacgtgc aggctgttta ataggggctg aacatgtcaa caactcatat |
| 1981 | gagtgtgaca tacccattgg tgcaggtata tgcgctagtt atcagactca gactaattct |
| 2041 | cctcggcggg cacgtagtgt agctagtcaa tccatcattg cctacactat gtcacttggt |
| 2101 | gcagaaaatt cagttgctta ctctaataac tctattgcca tacccacaaa ttttactatt |
| 2161 | agtgttacca cagaaattct accagtgtct atgaccaaga catcagtaga ttgtacaatg |
| 2221 | tacatttgtg gtgattcaac tgaatgcagc aatcttttgt tgcaatatgg cagtttttgt |
| 2281 | acacaattaa accgtgcttt aactggaata gctgttgaac aagacaaaaa cacccaagaa |
| 2341 | gtttttgcac aagtcaaaca aatttacaaa acaccaccaa ttaaagattt tggtggtttt |
| 2401 | aatttttcac aaatattacc agatccatca aaaccaagca agaggtcatt tattgaagat |
| 2461 | ctacttttca acaaagtgac acttgcagat gctggcttca tcaaacaata tggtgattgc |
| 2521 | cttggtgata ttgctgctag agacctcatt tgtgcacaaa agtttaacgg ccttactgtt |
| 2581 | ttgccacctt tgctcacaga tgaaatgatt gctcaataca cttctgcact gttagcgggt |
| 2641 | acaatcactt ctggttggac ctttggtgca ggtgctgcat tacaaatacc atttgctatg |
| 2701 | caaatggctt ataggtttaa tcgtattgga gttacacaga atgttctcta tgagaaccaa |
| 2761 | aaattgattg ccaaccaatt taatagtgct attggcaaaa ttcaagactc actttcttcc |
| 2821 | acagcaagtg cacttggaaa acttcaagat gtggtcaacc aaaatgcaca agctttaaac |
| 2881 | acgcttgtta aacaacttag ctccaatttt ggtgcaattt caagtgtttt aaatgatatc |
| 2941 | ctttcacgtc ttgacaaagt tgaggctgaa gtgcaaattg ataggttgat cacaggcaga |
| 3001 | cttcaaagtt tgcagacata tgtgactcaa caattaatta gagctgcaga aatcagagct |
| 3061 | tctgctaatc ttgctgctac taaaatgtca gagtgtgtac ttggacaatc aaaaagagtt |
| 3121 | gatttttgtg gaaagggcta tcatcttatg tccttccctc agtcagcacc tcatggtgta |
| 3181 | gtcttcttgc atgtgactta tgtccctgca caagaaaaga acttcacaac tgctcctgcc |
| 3241 | atttgtcatg atggaaaagc acactttcct cgtgaaggtg tctttgtttc aaatggcaca |
| 3301 | cactggtttg taacacaaag gaatttttat gaaccacaaa tcattactac agacaacaca |
| 3361 | tttgtgtctg gtaactgtga tcttgtaata ggaattgtca acaacacagt ttatgatcct |
| 3421 | ttgcaacctg aattagactc attcaaggag gagttagata aatattttaa gaatcataca |
| 3481 | tcaccagatg ttgatttagg tgacatctct ggcattaatg cttcagttgt aaacattcaa |
| 3541 | aaagaaattg accgcctcaa tgaggttgcc aagaatttaa atgaatctct catcgatctc |
| 3601 | caagaacttg gaaagtatga gcagtatata aaatggccat ggtacatttg gctaggtttt |
| 3781 | atagctggct tgattgccat agtaatggtg acaattatgc tttgctgtat gaccagttgc |
| 3661 | tgtagttgtc tcaagggctg ttgttcttgt ggatcctgct gcaaatttga tgaagacgac |
| 3721 | tctgagccag tgctcaaagg agtcaaatta cattacacat aa |

Coronaviruses use the spike glycoprotein to enter host cells, allowing them to deposit their genetic material into the host for production of new viral particles. The spike binds to host cell receptors, leading to a series of conformational changes that convert the pre-fusion spike structure into the post-fusion structure, which pulls the viral and host membranes together. Antibodies produced by the host immune system can bind to the spike and prevent these structure changes or prevent binding to the host receptor. This can block viral entry. Current COVID-19 vaccines work by exposing the host to the spike protein (either by direct injection of spike proteins, or injection of DNA or RNA that codes for the spike protein, which is then produced by the host). Unmodified spike proteins tend to be unstable, and readily undergo the transition to the postfusion structure. However, this is detrimental for immune response, since the antibodies need to bind the prefusion structure. Stabilizing the spike in the prefusion structure improves immune response, and many current COVID-19 vaccines use modifications to the spike amino acid sequence in order to produce spike proteins that are more stable in the pre-fusion form. These involve rigidification of a region of the spike known as the central helix (CH), which also changes interactions of the central helix with the receptor binding domain (RBD) of the spike. The change to these interactions has been reported to change the spike flexibility and motion of the receptor binding domain as compared to the true coronavirus spike. Many known antibodies bind to the receptor binding domain, and thus maintaining the original flexibility is expected to be important.

The S protein is highly conserved among all human coronaviruses (HCoVs) and is involved in receptor recognition, viral attachment, and entry into host cells. With a typical size of about 180-200 kDa, the S protein consists of an extracellular N-terminus, a transmembrane (TM) domain anchored in the viral membrane, and a short intracellular C-terminal segment. The S protein normally exists in a metastable, prefusion conformation; once the virus interacts with the host cell, extensive structural rearrangement of the S protein occurs, allowing the virus to fuse with the host cell membrane. The spikes are coated with polysaccharide molecules to camouflage them, evading surveillance of the host immune system during entry.

The total length of SARS-CoV-2 S is 1273 amino acids (aa) and consists of a signal peptide (amino acids 1-13) located at the N-terminus, the 51 subunit (14-685 residues), and the S2 subunit (686-1273 residues); the last two regions are responsible for receptor binding and membrane fusion, respectively. In the 51 subunit, there is an N-terminal domain (14-305 residues) and a receptor-binding domain (RBD, 319-541 residues); the fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) comprise the S2 subunit. S protein trimers visually form a characteristic bulbous, crown-like halo surrounding the viral particle. Based on the structure of coronavirus S protein monomers, the 51 and S2 subunits form the bulbous head and stalk region. The structure of the SARS-CoV-2 trimeric S protein has been determined by cryo-electron microscopy at the atomic level, revealing different conformations of the S RBD domain in opened and closed states and its corresponding functions (Huang. Y. Yang, C., Xu, Xf et al. Structural and functional properties of SARS-CoV-2 spike protein: potential antivirus drug development for COVID-19. Acta Pharmacol Sin 41, 1141-1149 (2020). doi.org/10.1038/s41401-020-0485-4).

Engineered Polypeptides

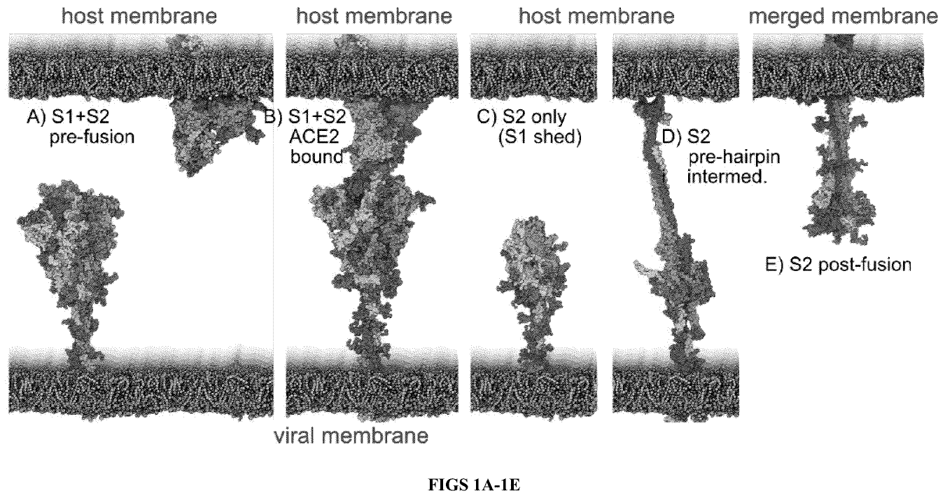

The compositions and methods described herein take a different approach to stabilizing the prefusion spike by introduction of a specifically designed disulfide bond that “staples” together the central helix and a region of the spike known as HR1. By preventing HR1 from detaching from CH, the prefusion spike structure is stabilized without rigidification of the central helix or changes to its interaction with the receptor binding domain. In certain embodiments, a double substitution through introduction of two cysteine amino acids at amino acid positions 970 and 999 (F970C+G999C) is provided. See, FIGS. 1A-1E. Positions 970 and 999 are otherwise highly conserved among known coronaviruses. This disulfide-stapled spike will be more stable in the prefusion form, allowing for a stable vaccine without the need for the stabilizing mutations that are currently in use.

Substitution with proline at the top of the central helix has been reported to change spike flexibility due to loss of interactions with the original amino acids at those locations. This may change the behavior of the modified spike as compared to the original protein, and thus when used as a vaccine it may not be an ideal molecule to train the immune system to recognize the unmodified spike on virions. The new design of spike proteins disclosed herein, which incorporate disulfide bridges as staples avoid these changes to flexibility, while still blocking the change from the prefusion to the postfusion spike.

Accordingly, the disclosure provides engineered polypeptides derived or modified from the spike (S) glycoprotein of coronaviruses, including SARS-CoV, MERS-CoV and SARS-CoV-2. The polypeptides comprise S sequences with modifications that stabilize the pre-fusion S1/S2 spike structure relative to the wild-type soluble S protein sequence of coronaviruses. The modifications include substitutions in the spike proteins which allowing for the formation of disulfide bridges. An example of a coronavirus spike amino acid sequence is the SARS-CoV-2 spike protein: NCBI Reference Sequence: YP_009724390.1, incorporated herein by reference in its entirety (SEQ ID NO: 3):

```
      1 mfvflvllpl vssqcvnltt rtqlppaytn sftrgvyypd kvfrssvlhs tqdlflpffs
    61 nvtwfhaihv sgtngtkrfd npvlpfndgv yfasteksni irgwifgttl dsktqslliv
  121 nnatnvvikv cefqfcndpf lgvyyhknnk swmesefrvy ssannctfey vsqpflmdle
  181 gkqgnfknlr efvfknidgy fkiyskhtpi nlvrdlpqgf saleplvdlp iginitrfqt
  241 llalhrsylt pgdsssgwta gaaayyvgyl qprtfllkyn engtitdavd caldplsetk
  301 ctlksftvek giyqtsnfrv qptesivrfp nitnlcpfge vfnatrfasv yawnrkrisn
  361 cvadysvlyn sasfstfkcy gvsptklndl cftnvyadsf virgdevrqi apgqtgkiad
  421 ynyklpddft gcviawnsnn ldskvggnyn ylyrlfrksn lkpferdist eiyqagstpc
  481 ngvegfncyf plqsygfqpt ngvgyqpyrv vvlsfellha patvcgpkks tnlvknkcvn
  541 fnfngltgtg vltesnkkfl pfqqfgrdia dttdavrdpq tleilditpc sfggvsvitp
  601 gtntsnqvav lyqdvnctev pvaihadqlt ptwrvystgs nvfqtragcl igaehvnnsy
  661 ecdipigagi casyqtqtns prrarsvasq siiaytmslg aensvaysnn siaiptnfti
  721 svtteilpvs mtktsvdctm yicgdstecs nillqygsfc tqlnraltgi aveqdkntqe
  781 vfaqvkqiyk tppikdfggf nfsqilpdps kpskrsfied llfnkvtlad agfikqygdc
  841 1gdiaardli caqkfngltv lpplltdemi aqytsallag titsgwtfga gaalqipfam
  901 qmayrfngig vtqnvlyenq klianqfnsa igkiqdslss tasalgklqd vvnqnaqaln
  961 tlvkqlssnf gaissvlndi 1srldkveae vqidrlitgr lqslqtyvtq qliraaeira
1021 sanlaatkms ecvlgqskrv dfcgkgyhlm sfpqsaphgv vflhvtyvpa qeknfttapa
1081 ichdgkahfp regvfvsngt hwfvtqrnfy epqiittdnt fvsgncdvvi givnntvydp
1141 lqpeldsfke eldkyfknht spdvdlgdis ginasvvniq keidrlneva knlneslidl
1201 qelgkyeqyi kwpwyiwlgf iagliaivmv timlccmtsc csclkgccsc gscckfdedd
1261 sepvlkgvkl hyt
```

The F at position 970 and the G at position 999 of SEQ ID NO: 3 are underlined above. The S1 subunit corresponds to residues 14-685 of SEQ ID NO: 3, and the S2 subunit corresponds to residues 686-1273 of SEQ ID NO: 3. As detailed herein, some of S1/S2 polypeptides of the present disclosure comprise mutations that can enhance the stability of the S1/S2 structure prior to fusion. These include mutations that stabilize the prefusion S1/S2 complex. In some embodiments, the mutations inactivate the S1/S2 cleavage site.

The engineered proteins provided herein, comprise a coronavirus S1/S2 prefusion spike peptide or protein which has been stabilized by engineering one or more disulfide bridges as disclosed herein. The disulfide bonds maintain the S1/S2 prefusion complex, whereas the wild-type coronavirus sheds the 51 leaving a metastable S2 (see, FIGS. 1A-1E). In embodiments, the coronavirus S1/S2 prefusion spike peptide or protein comprises an amino acid sequence, S₉₆₇SNF₉₇₀GAISSVLNDILSRLDKVEAEVQIDRLITG₉₉₉R₁₀₀₀ (SEQ ID NO: 1), wherein phenylalanine at position 970 (F970) and glycine at position 999 (G999) of SEQ ID NO: 1 are substituted with cysteines. In other embodiments, a coronavirus S1/S2 prefusion spike peptide or protein comprises an F to C (Phe to Cys) substitution and a G to C (Gly to Cys) substitution at positions corresponding to F970 and G999 of SARS-CoV-2 spike protein provided in SEQ ID NO: 3. In certain embodiments, the coronavirus S1/S2 prefusion spike peptide or protein comprises an engineered disulfide bond at paired cysteine substitutions at positions corresponding to F970 and G999 of SEQ ID NO 3, including e.g., F1060 and G1089; F1036 and G1065; F1044 and G1073; F952 and G981; F839 and G868; F843 and G872; F1064 and G1093; F1020 and G1049.

The disclosure should also be construed to include any form of a protein having substantial homology to a coronavirus S1/S2 prefusion spike protein. Preferably, a protein which is “substantially homologous” about 70% homologous, preferably about 80% homologous, more preferably about 90% homologous, even more preferably, about 95% homologous, and even more preferably about 99% homologous to amino acid sequence of a coronavirus S1/S2 prefusion spike peptide or protein disclosed herein.

In another aspect, an engineered peptide comprises an amino acid sequence having at least 80%, at least 85%, at least 90%, at least 95%, at least 99%, or 99.9% amino acid sequence identity to S₉₆₇SNF₉₇₀GAISSVLNDILSRLDKVEAEVQIDRLITG₉₉₉R₁₀₀₀ (SEQ ID NO: 1). In certain embodiments, the engineered peptide comprises an amino acid sequence having at least 80%, at least 85%, at least 90%, at least 95%, at least 99%, or 99.9% amino acid sequence identity to S₉₆₇SNF₉₇₀GAISSVLNDILSRLDKVEAEVQIDRLITG₉₉₉R₁₀₀₀ (SEQ ID NO: 1) and includes one or more disulfide bridges to retain an S1/S2 prefusion spike protein complex.

In certain aspects, a coronavirus S1/S2 prefusion spike peptide comprises an amino acid sequence having at least 80%, at least 85%, at least 90%, at least 95%, at least 99%, or 99.9% amino acid sequence identity to a wild-type coronavirus S1/S2 prefusion peptide, e.g. as in SEQ ID NO: 3. In certain embodiments, a coronavirus S1/S2 prefusion spike peptide comprises an amino acid sequence having at least 80%, at least 85%, at least 90%, at least 95%, at least 99%, or 99.9% amino acid sequence identity to a wild-type coronavirus S1/S2 prefusion peptide, e.g., as in SEQ ID NO: 3, and includes one or more disulfide bridges to retain an S1/S2 prefusion spike protein complex.

In certain aspects, a coronavirus S1/S2 prefusion spike peptide, e.g., as in SEQ ID NO: 3, comprises an amino acid sequence having at least 80%, at least 85%, at least 90%, at least 95%, at east 99%, or 99.9% amino acid sequence identity to S₉₆₇SNF₉₇₀GAISSVLNDILSRLDKVEAEVQIDRLITG₉₉₉R₁₀₀₀ (SEQ ID NO: 1). In certain embodiments, a coronavirus S1/S2 prefusion spike peptide comprises an amino acid sequence having at least 80%, at least 85%, at least 90%, at least 95%, at least 99%, or 99.9% amino acid sequence identity to S₉₆₇SNF₉₇₀GAISSVLNDILSRLDKVEAEVQIDRLITG₉₉₉R₁₀₀₀ (SEQ ID NO: 1) and includes one or more disulfide bridges to retain an S1/S2 prefusion spike protein complex.

The engineered peptides may alternatively be made by recombinant means or by cleavage from a longer polypeptide. The composition of a peptide may be confirmed by amino acid analysis or sequencing. The variants of the peptides according to the present disclosure may be (i) one in which one or more of the amino acid residues are substituted with a conserved or nonconserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, or different from the original sequence in less than 40% of residues per segment of interest, or different from the original sequence in less than 25% of residues per segment of interest, or different by less than 10% of residues per segment of interest, or different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction. The peptides of the disclosure may include unnatural amino acids formed by post-translational modification or by introducing unnatural amino acids during translation. A variety of approaches are available for introducing unnatural amino acids during protein translation.

The peptides of the disclosure can also be conjugated to each other to form dimers, trimers, etc. Single chain peptide linkers, comprised of for example, from one to twenty amino acids joined by peptide bonds, can be used. In certain embodiments, the amino acids are selected from the twenty naturally occurring amino acids. In certain embodiments, the amino acids are selected from the twenty naturally occurring amino acids, unnatural amin acids, modified amino acids, analogs or combinations thereof. In certain other embodiments, one or more of the amino acids are selected from glycine, alanine, proline, asparagine, glutamine and lysine. In other embodiments, the linker is a chemical linker. In certain embodiments, linker is a single chain peptide with an amino acid sequence with a length of at least 25 amino acids, or with a length of 32 to 50 amino acids.

A peptide or protein of the disclosure may be conjugated with other molecules to prepare fusion proteins. This may be accomplished, for example, by the synthesis of N-terminal or C-terminal fusion proteins. In certain embodiments, the nucleic acid sequences conjugation may be performed using a variety of chemical linkers. For example, an S1/S2 prefusion spike peptide can be conjugated with one or more S1/S2 prefusion spike peptides, or conjugated to other molecules, using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). The linker may be a “cleavable linker” facilitating release of the stabilized S1/S2 prefusion complex into the subject's blood stream or tissues dependent on the mode of administration. For example, an acid-labile linker, peptidase-sensitive linker, photolabile linker, dimethyl linker or disulfide-containing linker (Chari et al, Cancer Res. 52: 127-131 (1992); U.S. Pat. No. 5,208,020) may be used.

Covalent conjugation can either be direct or via a linker. In certain embodiments, direct conjugation is by construction of a protein fusion (i.e., by genetic fusion of two or more genes or nucleic acid sequences and expressed as a single protein. In certain embodiments, direct conjugation is by formation of a covalent bond between a reactive group on one of the two portions of the S1/S2 complex and a corresponding group or acceptor on a second molecule of interest. In certain embodiments, direct conjugation is by modification (i.e., genetic modification) of one of the two molecules to be conjugated to include a reactive group (as non-limiting examples, a sulfhydryl group or a carboxyl group) that forms a covalent attachment to the other molecule to be conjugated under appropriate conditions. Methods for covalent conjugation of nucleic acids to proteins are also known in the art (i.e., photocrosslinking, see, e.g., Zatsepin et al. Russ. Chem. Rev. 74: 77-95 (2005)).

In certain embodiments, at least one or more peptides or proteins of the disclosure are conjugated with one or more biomolecules of interest. Examples of biomolecules include cytokines, growth factors, viral antigens, polynucleotides, oligonucleotides, hormones, enzymes, checkpoint proteins, an antigen, an antibody, a transcription factor, a receptor, a ligand, immunoglobulins, immunoglobulin fragments, a fluorescent protein, etc. The length of the biomolecule, e.g., peptide of interest may vary as long as the amount of the targeted biomolecule, e.g., a peptide produced is significantly increased when expressed in the form of a fusion peptide/chimeric molecule.

Examples of enzymes include enzymes such as lipase, protease, steroid synthesizing enzyme, kinase, phosphatase, xylanase, esterase, methylase, demethylase, oxidase, reductase, cellulase, aromatase, Carnauba, transglutaminase, glycosidase, and chitinase. Growth factors include, for example, epithelial growth factor (EGF), insulin-like growth factor (IGF), transforming growth factor (TGF), nerve growth factor (NGF), brain derived neurotrophic factor (BDNF) (VEGF), granulocyte colony stimulating factor (G-CSF), granulocyte macrophage colony stimulating factor (GM-CSF), platelet derived growth factor (PDGF), erythropoietin (EPO), thrombopoietin, Pre-eukaryotic cell growth factor (FGF), hepatocyte growth factor (HGF). Examples of the hormone include insulin, glucagon, somatostatin, growth hormone, parathyroid hormone, prolactin, leptin and calcitonin. Examples of cytokines include interleukin, interferon (IFN alpha, IFN beta, IFN gamma), tumor necrosis factor (TNF). Blood proteins include, for example, thrombin, serum albumin, Factor VII, Factor VII, Factor X, Factor X, tissue plasminogen activator. Antibody proteins include for example, F (ab′)₂, Fc, Fc fusion protein, heavy chain (H chain), light chain (L chain), short chain Fv scFv), sc(Fv)₂, disulfide-linked Fv (sdFv), Diabodies.

Immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR.

Antigens may be appropriately selected depending on the subject of the immunological response, for example, a protein derived from one or more coronavirus variants.

The peptides or proteins of the disclosure may be combined with a secretory signal peptide functioning in the host cell for secretory production. When the yeast is used as a host, the secretory signal peptide can be exemplified by an invertase secretion signal. In certain embodiments, the secretory signal is obtained from two or more different sources. Various sources include, for example, Bacillus species, Lactococcus lactis, Streptomyces, or Corynebacterium. Other signal sequences include, for example, human IL-2, human chymotrypsin, human interferon gamma, etc.

In certain embodiments, the peptides or proteins of the disclosure may be added with a transport signal peptide such as an endoplasmic reticulum residual signal peptide or a liquid phase transition signal peptide for expression in a specific cell compartment.

Proteins or peptides may be made by any technique known to those of skill in the art, including the expression of proteins, polypeptides or peptides through standard molecular biological techniques, the isolation of proteins or peptides from natural sources, in vitro translation, the chemical synthesis of proteins or peptides or can be genetically produced. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield R B: Solid phase peptide synthesis. I. The synthesis of a tetrapeptide. J. Am. Chem. Soc. 85:2149-54, 1963). In certain embodiments, neoantigenic peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

Nucleic Acid Compositions

In certain embodiments, a vector encodes a coronavirus S1/S2 prefusion spike peptide comprising at least a 50% (such as at least about 55%, 60%, 65%, 70%, 75%, 80%, 85%, 86%, 87%, 88%, 89%, 90%, 91%, 92%, 93%, 94%, 95%, 96%, 97%, 98%, 99%, or greater) sequence identity to S₉₆₇SNF₉₇₀GAISSVLNDILSRLDKVEAEVQIDRLITG₉₉₉R₁₀₀₀ (SEQ ID NO: 1).

In certain embodiments, a vector encodes a coronavirus S1/S2 prefusion spike peptide comprising at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, a vector encodes a coronavirus S1/S2 prefusion spike peptide comprising at least an 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, a vector encodes a coronavirus S1/S2 prefusion spike peptide comprising at least a 90%, 91%, 92%, 93%, 94%, 95%, 96%, 97%, 98%, or 99%, sequence identity to SEQ ID NO: 1. In certain embodiments, a vector encodes a coronavirus S1/S2 prefusion spike peptide comprising an amino acid sequence, S₉₆₇SNF₉₇₀GAISSVLNDILSRLDKVEAEVQIDRLITG₉₉₉R₁₀₀₀ (SEQ ID NO: 1), wherein phenylalanine at position 970 (F970) and glycine at position 999 (G999) of SEQ ID NO: 1 are substituted with cysteines. 8, 11 or 14.

Genetic constructs or vectors comprise a nucleotide sequence that encodes a desired protein operably linked to regulatory elements needed for gene expression. Accordingly, incorporation of the DNA or RNA molecule into a living cell results in the expression of the DNA or RNA encoding the desired protein and thus, production of the desired protein. The proteins and peptides of the present disclosure can be produced by a general genetic engineering technique. For example, a recombinant vector encoding for a stabilized coronavirus S1/S2 prefusion spike protein. The recombinant vector of the present disclosure is not particularly limited as long as the nucleic acid sequences are inserted into the vector so that it can be expressed in a host cell into which the vector is introduced. The vector is not particularly limited as long as it is replicable in the host cell, and examples thereof include plasmid DNA and viral DNA. The regulatory elements necessary for gene expression of a DNA molecule include: a promoter, an initiation codon, a stop codon, and a polyadenylation signal. In addition, enhancers are often required for gene expression. It is necessary that these elements be operable linked to the sequence that encodes the desired proteins and that the regulatory elements are operably in the individual to whom they are administered.

Initiation codons and stop codon are generally considered to be part of a nucleotide sequence that encodes the desired protein. However, it is necessary that these elements are functional in the individual to whom the gene construct is administered. The initiation and termination codons must be in frame with the coding sequence.

The molecule that encodes a desired protein may be DNA or RNA which comprise a nucleotide sequence that encodes the desired protein. These molecules may be cDNA, genomic DNA, synthesized DNA or a hybrid thereof or an RNA molecule such as mRNA. Accordingly, as used herein, the terms “DNA construct”, “genetic construct”, “nucleotide sequence”, nucleic acid” are meant to refer to both DNA and RNA molecules.

When taken up by a cell, the genetic construct which includes the nucleotide sequence encoding the desired protein operably linked to the regulatory elements may remain present in the cell as a functioning extrachromosomal molecule or it may integrate into the cell's chromosomal DNA. DNA may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also contemplated to provide the genetic construct as a linear minichromosome including a centromere, telomeres and an origin of replication.

Accordingly, in certain embodiments, the present disclosure includes a vector comprising one or more cassettes comprising a nucleic acid sequence encoding the proteins and peptides of the disclosure. The vector can be any vector that is known in the art and is suitable for expressing the desired expression cassette. A number of vectors are known or can be designed to be capable of mediating transfer of gene products to mammalian cells, as is known in the art and described herein. In certain aspects, a vector refers to a nucleic acid polynucleotide to be delivered to a host cell, either in vitro or in vivo. In some embodiments, one or more cassettes are provided on a single vector. In some embodiments, one or more cassettes are provided on a two or more vectors. In some embodiments, cassettes are provided by one or more vectors comprising an isolated nucleic acid encoding one or more elements of a stabilized coronavirus S1/S2 prefusion spike peptide. In some embodiments, the cassettes are provided by one or more vectors comprising an isolated nucleic acid encoding one or more components a stabilized coronavirus S1/S2 prefusion spike peptide. In some instances, the expression of natural or synthetic nucleic acids encoding an RNA and/or peptide is typically achieved by operably linking a nucleic acid encoding the RNA and/or peptide or portions thereof to a promoter and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The isolated nucleic acids of the disclosure can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Additional promoter elements, e.g., enhancers, regulate the frequency of transcriptional initiation. In some embodiments, the vector also includes conventional control elements which are operably linked to the transgene in a manner which permits its transcription, translation and/or expression in a cell transfected with the plasmid vector or infected with the virus comprising a nucleic acid comprising the described cassettes or compositions. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and can be utilized.

Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The selection of appropriate promoters can readily be accomplished. In certain aspects, one would use a high expression promoter. Promoters and polyadenylation signals used must be functional within the cells of the individual. The promoter used in the vector may be appropriately selected depending on the host cell into which the vector is introduced. For example, when expressed in yeast, the GAL1 promoter, the PGK1 promoter, the TEF1 promoter, the ADH1 promoter, the TPI1 promoter, the PYK1 promoter and the like can be used. When expressed in plants, Cauliflower Mosaic Virus 35S promoter, rice actin promoter, corn ubiquitin promoter, lettuce ubiquitin promoter, and the like can be used. When expressed in Escherichia coli, T7 promoter and the like can be used. In the case of expression in Brevibacillus, P2 promoter and P22 promoter and the like can be mentioned. Inducible promoter. For example, in addition to lac, tac and trc which are inducible by IPTG, trp which can be induced by IAMINO ACIDS, ara which can be induced by L-arabinose, Pzt-1 which can be induced by using tetracycline, A P L promoter inducible at high temperature (42° C.), and a promoter of cspA gene, which is one of cold shock genes. Other examples of promoters useful in the production of a genetic vaccine for humans, include but are not limited to promoters from Simian Virus 40 (SV40, Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein. Examples of polyadenylation signals useful to practice the present disclosure, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, is used.

One example of a suitable promoter is the CAG promoter or the immediate early cytomegalovirus (CMV) promoter sequence. This promoter sequence is a strong constitutive promoter sequence capable of driving high levels of expression of any polynucleotide sequence operatively linked thereto. In certain embodiments, the Rous sarcoma virus (RSV) and MMT promoters are also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication.

Another example of a suitable promoter is Elongation Growth Factor-1α (EF-1α). However, in some embodiments, other constitutive promoter sequences are used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosed should not be limited to the use of constitutive promoters. Inducible promoters are also contemplated as part of the disclosed. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. In some instances, enhancers are located upstream or downstream of the gene it regulates. In some instances, enhancers are also tissue-specific to enhance transcription in a specific cell or tissue type. In some embodiments, the vector of the present disclosure comprises one or more enhancers to boost transcription of the gene present within the vector. In some instances, the expression of the nucleic acid and/or protein, the expression vector to be introduced into a cell can also contain either a selectable marker gene or a reporter gene or both to facilitate identification and selection of expressing cells from the population of cells sought to be transfected or infected through viral vectors. In other embodiments, the selectable marker is carried on a separate piece of DNA and used in a co-transfection procedure. Both selectable markers and reporter genes can be flanked with appropriate regulatory sequences to enable expression in the host cells. Useful selectable markers include, for example, antibiotic-resistance genes, such as neo and the like.

If necessary, a terminator sequence may also be included depending on the host cell. The recombinant vector of the present disclosure can be produced, for example, by digesting a DNA construct with a suitable restriction enzyme, or adding a restriction enzyme site by PCR, and inserting the vector into a restriction enzyme site or a multicloning site.

Vaccines and Immunological Compositions

In certain embodiments, the peptides identified according to the present disclosure are used in a vaccine or immunological composition to prevent or treat a coronavirus infection. The term “vaccine” or “immunological composition” are used interchangeably and are meant to refer in the present context to a pooled sample of one or more antigenic peptides, for example at least one, at least two, at least three, at least four, at least five, or more antigenic peptides. A “vaccine” is to be understood as including a protective vaccine, which is a composition for generating immunity for the prophylaxis and/or treatment of diseases. A protective vaccine may be formulated with antigenic epitopes specific for a coronavirus. Accordingly, vaccines are medicaments which comprise antigens and are intended to be used in humans or animals for generating specific defense and protective substance by vaccination. A “vaccine composition” can include a pharmaceutically acceptable excipient, carrier or diluent.

The vaccine may include one or more peptides identified according to the present disclosure. For example, 1 to 10 peptides. Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

In Vivo Peptide/Polypeptide Synthesis

The present disclosure also contemplates the use of nucleic acid molecules as vehicles for delivering peptides/polypeptides to the subject in need thereof, in vivo, in the form of, e.g., DNA/RNA vaccines.

In certain embodiments, antigens may be administered to a patient in need thereof by use of a plasmid. These are plasmids which usually consist of a strong viral promoter to drive the in vivo transcription and translation of the gene (or complementary DNA) of interest (Mor, et al., (1995), Journal Immunol. 155 (4): 2039-2046). Intron A may sometimes be included to improve mRNA stability and hence increase protein expression (Leitner et al. (1997), Journal Immunol. 159 (12): 6112-6119). Plasmids also include a strong polyadenylation/transcriptional termination signal, such as bovine growth hormone or rabbit beta-globulin polyadenylation sequences Multi cistronic vectors are sometimes constructed to express more than one immunogen, or to express an immunogen and an immunostimulatory protein.

One way of enhancing protein expression is by optimizing the codon usage of pathogenic mRNAs for eukaryotic cells. Another consideration is the choice of promoter. Such promoters may be the SV40 promoter or Rous Sarcoma Virus (RSV). Plasmids may be introduced into animal tissues by a number of different methods. The two most used approaches are injection of DNA in saline, using a standard hypodermic needle, and gene gun delivery. Injection in saline is normally conducted intramuscularly (EVI) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose. Immune responses to this method of delivery can be affected by many factors, including needle type, needle alignment, speed of injection, volume of injection, muscle type, and age, sex and physiological condition of the animal being injected.

Alternative delivery methods may include aerosol instillation of naked DNA on mucosal surfaces, such as the nasal and lung mucosa, and topical administration of pDNA to the eye and vaginal mucosa. Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Ex vivo Cytosolic Delivery of Functional Macromolecules to Immune Cells, Sharei et al., PLOS ONE DOI: 10.1371/journal.pone.O1 18803 Apr. 13, 2015).

In certain embodiments, a vaccine or immunogenic composition may include separate DNA plasmids encoding, for example, one or more a coronavirus S1/S2 prefusion spike peptides/polypeptides as identified in according to the disclosure. As discussed herein, the exact choice of expression vectors can depend upon the peptide/polypeptides to be expressed and is well within the skill of the ordinary artisan. The expected persistence of the DNA constructs (e.g., in an episomal, non-replicating, non-integrated form in the muscle cells) is expected to provide an increased duration of protection.

One or more antigenic peptides of the disclosure may be encoded and expressed in vivo using a viral based system (e.g., an adenovirus system, an adeno associated virus (AAV) vector, a poxvirus, or a lentivirus). In one embodiment, the vaccine or immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7). Plasmids that can be used for adeno associated virus, adenovirus, and lentivirus delivery have been described previously. The peptides and polypeptides of the disclosure can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the disclosure. Upon introduction into an acutely or chronically infected host or into a noninfected host, the vector expresses the immunogenic peptide, and thereby elicits a host CTL response.

Among vectors that may be used in the practice of the disclosure, integration in the host genome of a cell is possible with retrovirus gene transfer methods, often resulting in long term expression of the inserted transgene. In certain embodiments, the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the disclosure). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the disclosure include those based upon murine leukemia virus (MuLV), gibbon ape leukemia virus (GaLV), Simian Immunodeficiency virus (SIV), human immunodeficiency virus (HIV), and combinations thereof.

Also useful is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the disclosure contemplates amongst vector(s) useful in the practice of the disclosure: viral vectors, including retroviral vectors and lentiviral vectors.

One of skill in the art can determine suitable dosage. Suitable dosages for a virus can be determined empirically. Also useful in the practice of the disclosure is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848).

The adenovirus vector used can be selected from the group consisting of the Ad5, Ad35, Adl 1, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285). Ad35 vectors are described in U.S. Pat. Nos. 6,974,695, 6,913,922, and 6,869,794. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present disclosure. Furthermore, “gutless” adenovirus vectors, in which all viral genes are deleted, can also be used in accordance with the present disclosure. Such vectors require a helper virus for their replication and require a special human 293 cell line expressing both E1a and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present disclosure and can even be used for co-delivery of a large number of heterologous inserts/genes.

In one embodiment, the viral vector is an adenovirus vector, an adeno-associated viral vector (AAV), pseudotyped AAVs, or derivatives thereof. The adeno-associated viral vector comprises AAV serotypes 1, 2, 3, 4, 5, 6, 7, 8, 9, 10, 11, 12, 13, 14, 15, 16, DJ or DJ/8. In one embodiment, the AAV can be AAV1, AAV2, AAV5 or any combination thereof. One can select the AAV with regard to the cells to be targeted, e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

In another embodiment effectively activating a cellular immune response for a immunogenic composition can be achieved by expressing the relevant antigens in a vaccine or immunogenic composition in a non-pathogenic microorganism. Well-known examples of such microorganisms are Mycobacterium bovis BCG, Salmonella and Pseudomona (See, U.S. Pat. No. 6,991,797). In another embodiment a Poxvirus is used in the immunogenic composition. These include orthopoxvirus, avipox, vaccinia, MVA, NYVAC, canarypox, ALVAC, fowlpox, TROVAC, etc. (see e.g., Verardi et al., Hum Vaccin Immunother. 2012 July; 8(7):961-70; and Moss, Vaccine. 2013; 31(39): 4220-4222).

Poxviruses that may be used in the practice of the disclosure, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC. 1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature.

In one embodiment recombinant viral particles of the vaccine or immunogenic composition are administered to patients in need thereof. Dosages of expressed neoantigen can range from a few to a few hundred micrograms, e.g., 5 to 500 μg. The vaccine or immunogenic composition can be administered in any suitable amount to achieve expression at these dosage levels. The viral particles can be administered to a patient in need thereof or transfected into cells in an amount of about at least 10³ pfu; thus, the viral particles are preferably administered to a patient in need thereof or infected or transfected into cells in at least about 10⁴ pfu to about 10⁶ pfu; however, a patient in need thereof can be administered at least about 10⁸ pfu or at least about 10⁷ pfu to about 10⁹ pfu. Doses as to NYVAC are applicable as to ALVAC, MVA, MVA-BN, and avipoxes, such as canarypox and fowlpox.

Combination Therapies

The compositions of the disclosure can also be administered to a subject in combination with one or more other therapies, termed herein “combination therapies”. The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

In certain embodiments, the proteins/peptides embodied herein are administered to a patient in combination with one or more other anti-viral agents or therapeutics. Examples include any molecules that are used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, an interferon, an amino acid analog, a nucleoside analog, an integrase inhibitor, a protease inhibitor, a polymerase inhibitor, and a transcriptase inhibitor. Other examples of anti-viral agents include acyclovir (ACV), ganciclovir (GCV), famcyclovir, penciclovir, foscarnet, ribavirin, zalcitabine (ddC), zidovudine (AZT), stavudine (D4T), Iarnivudine (3TC), didanosine (ddl), cytarabine, dideoxyadenosine, edoxudine, floxuridine, idozuridine, inosine pranobex, 2′-deoxy-5-(methylamino)uridine, trifluridine or vidarabine.

In certain embodiments, the protein/peptide compositions embodied herein are administered with one or more compositions comprising a therapeutically effective amount of a non-nucleoside reverse transcriptase inhibitor (NNRTI) and/or a nucleoside reverse transcriptase inhibitor (NRTI), analogs, variants or combinations thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, a composition comprises a therapeutically effective amount of at least one NNRTI or a combination of NNRTI's, analogs, variants or combinations thereof. In certain embodiments, the NNRTI is rilpivirine. In certain embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, the composition comprises a therapeutically effective amount of at least one or a combination of NRTI's, analogs, variants or combinations thereof.

In certain embodiments, the protein/peptide compositions embodied herein are administered with one or more cell based therapies. For example, a T cell expressing a chimeric antigen receptor which specifically recognizes a coronavirus antigen can be generated. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In other embodiments, a subject's immune cells are cultured ex vivo with coronavirus antigens and then re-infused into the patient. In various embodiments the cell-based therapy is the adoptive transfer of allogenic donor-derived cells.

It is contemplated that other agents may be used in combination with the compositions provided herein to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines.

When administered as a combination, the therapeutic agents, e.g., a stabilized coronavirus S1/S2 prefusion spike protein can be formulated as separate compositions that are given at the same time or different times, or the therapeutic agents can be given as a single composition.

Pharmaceutical Compositions/Methods of Delivery

In certain embodiments, a pharmaceutical composition comprises an effective amount of one or more antigenic peptides as described herein (including a pharmaceutically acceptable salt, thereof), optionally in combination with a pharmaceutically acceptable carrier, excipient or additive. The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. The liposomes will be targeted to and taken up selectively by the organ. The present disclosure also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., analgesic agents.

Administration of the compositions of this disclosure may be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g., tablets and sustained release capsules, and may be prepared by any methods well known in the art of pharmacy.

The pharmaceutical compositions may also comprise additional ingredients. As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the disclosure are known in the art and described, for example in Genaro, ed. (1985, Remington's Pharmaceutical Sciences, Mack Publishing Co., Easton, PA), which is incorporated herein by reference.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

Liquid suspensions may be prepared using conventional methods to achieve suspension the composition of the disclosure in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

Methods of Treatment

The present disclosure provides a method of treating or preventing a coronavirus infection. In some embodiments, the method comprises administering to a subject infected with a coronavirus, comprises administering to the subject a pharmaceutical composition comprising a therapeutically effective amount of a coronavirus S1/S2 prefusion spike peptide or expression vector encoding the coronavirus S1/S2 prefusion spike peptide, wherein the coronavirus S1/S2 prefusion spike peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1). In certain embodiments, phenylalanine at position 970 (F970) and glycine at position 999 (G999) of SEQ ID NO: 1 are substituted with cysteines. In certain embodiments, the method of treating or preventing a coronavirus infection in a subject, further comprises administering to the subject an agent or vaccine. In certain embodiments, the agent comprises an anti-viral agent, an immunomodulatory agent, an antibody, an antibody fragment, a chemotherapeutic agent, or a biological agent.

Kits

Kits are also contemplated. In one aspect, a kit comprises an engineered peptide comprises a coronavirus S1/S2 prefusion spike peptide sequence having one or more amino acid substitutions, mutations, deletions, insertions or combinations thereof. In certain embodiments, the coronavirus S1/S2 prefusion spike peptide sequence comprises two amino acid substitutions to form a disulfide bridge between the two substituted amino acids. In certain embodiments, the two amino acids are substituted at conserved amino acid positions of the coronavirus S1/S2 prefusion spike peptide sequence. In certain embodiments, the two amino acids comprise cysteine. In certain embodiments, the coronavirus S1/S2 prefusion spike peptide comprises peptide having a 90% sequence identity to S₉₆₇SNF₉₇₀GAISSVLNDILSRLDKVEAEVQIDRLITG₉₉₉R₁₀₀₀ (SEQ ID NO: 1). In certain embodiments, the coronavirus S1/S2 prefusion spike peptide comprises an amino acid sequence comprising S₉₆₇SNF₉₇₀GAISSVLNDILSRLDKVEAEVQIDRLITG₉₉₉R₁₀₀₀ (SEQ ID NO: 1). In certain embodiments, the coronavirus S1/S2 prefusion spike peptide sequence comprises a cysteine substitution at amino acid position 970. In certain embodiments, the coronavirus S1/S2 prefusion spike peptide sequence comprises a cysteine substitution at amino acid position 999. In certain embodiments, the coronavirus S1/S2 prefusion spike peptide sequence comprises a cysteine substitution at amino acid positions 970 and 999. In certain embodiments, the cysteines at amino acid positions 970 and 999 form a disulfide bridge.

In one aspect, a kit comprises a stabilized coronavirus S1/S2 prefusion spike protein comprising an amino acid sequence, SEQ ID NO: 1, wherein phenylalanine at position 970 (F970) and glycine at position 999 (G999) of SEQ ID NO: 1 are substituted with cysteines.

In one aspect, a kit comprises an engineered protein comprising a stabilized coronavirus S1/S2 prefusion spike protein comprising an engineered disulfide bond comprising paired cysteine substitutions at positions corresponding to: F970 and G999; F1060 and G1089; F1036 and G1065; F1044 and G1073; F952 and G981; F839 and G868; F843 and G872; F1064 and G1093; F1020 and G1049.

In one aspect, a kit comprises an expression vector encoding a coronavirus S1/S2 prefusion spike peptide comprises a 90% sequence identity to S₉₆₇SNF₉₇₀GAISSVLNDILSRLDKVEAEVQIDRLITG₉₉₉R₁₀₀₀ (SEQ ID NO: 1). In certain embodiments, the coronavirus S1/S2 prefusion spike peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1). In certain embodiments, phenylalanine at position 970 (F970) and glycine at position 999 (G999) of SEQ ID NO: 1 are substituted with cysteines. In certain embodiments, the cysteines at amino acid positions 970 and 999 form a disulfide bridge.

In some embodiments, the kit can further comprise at least one reagent for use in one or more embodiments of the methods described herein. Reagents that can be provided in the kit can include at least one or more of the following: a hybridization reagent, a purification reagent, an immobilization reagent, an imaging agent, a cell permeabilization agent, a blocking agent, a cleaving agent for the cleavable linker, and any combinations thereof.

In some embodiments, the kit can further include a computer-readable (non transitory) storage medium in accordance with one or more embodiments described herein. For example, in one embodiment, the computer-readable (non-transitory) storage medium included in the kit can provide instructions to determine the presence or expression levels of one or more target molecules in a sample. The computer-readable (non-transitory) storage medium can be in a CD, DVD, and/or USB drive.

In all such embodiments of the aspect, the kit includes the necessary packaging materials and information material therein to store and use said kits. The informational material can be descriptive, instructional, marketing or other material that relates to the methods described herein and/or the use of an agent(s) described herein for the methods described herein.

The informational material of the kits is not limited in its form. In many cases, the informational material, e.g., instructions, is provided in printed matter, e.g., a printed text, drawing, and/or photograph, a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In all embodiments of the aspects described herein, the kit will typically be provided with its various elements included in one package, e.g., a fiber-based, e.g., a cardboard, or polymeric, e.g., a styrofoam box. The enclosure can be configured so as to maintain a temperature differential between the interior and the exterior, e.g., it can provide insulating properties to keep the reagents at a preselected temperature for a preselected time. The kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit usage forms of target probes described herein. For example, the kit includes a plurality of syringes, ampules, foil packets, or blister packs, each containing a single unit usage of target probes described herein. The containers of the kits can be airtight, waterproof (e.g., impermeable to changes in moisture or evaporation), and/or lighttight.

Screening Assays

In certain aspects, the disclosure features methods for identifying agents or compounds useful in inhibiting coronavirus infections, such compounds having potential therapeutic use in the treatment of, for example, SARS-Cov-2. Accordingly, in exemplary aspects the disclosure features methods of identifying compounds useful in inhibiting coronavirus infections, the methods featuring screening or assaying for compounds that inhibit binding of the S1/S2 spike protein and host cell receptors, e.g., ACE2. In exemplary aspects, the methods comprise: providing an indicator composition comprising a coronavirus S1/S2 prefusion spike peptide or biologically active portions thereof; contacting the indicator composition with each member of a library of test compounds; and selecting from the library of test compounds a compound of interest that decreases the interaction of a coronavirus S1/S2 prefusion spike peptide and host receptor. In other aspects, the coronavirus S1/S2 prefusion spike peptide is used to generate antibodies or active fragments thereof which specifically bind to a coronavirus S1/S2 prefusion spike peptide.

In another aspect, a method of identifying candidate therapeutic agents for preventing or treating a coronavirus infection comprises contacting a substrate comprising: (i) a nucleic acid molecule encoding a peptide having a 90% amino acid sequence identity to S₉₆₇SNF₉₇₀GAISSVLNDILSRLDKVEAEVQIDRLITG₉₉₉R₁₀₀₀ (SEQ ID NO: 1); or, (ii) a peptide having a 90% amino acid sequence identity to S₉₆₇SNF₉₇₀GAISSVLNDILSRLDKVEAEVQIDRLITG₉₉₉R₁₀₀₀ (SEQ ID NO: 1); or, (iii) a nucleic acid molecule encoding a peptide comprising at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1), with a candidate therapeutic agent; or, (iv) a peptide comprising at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1), with a candidate therapeutic agent; or, (v) a cell comprising any one of nucleic acids or peptides of (i)-(iv); and conducting an assay for an output value. In certain embodiments, the assay comprises: immunoassays, Southern blots, Western blots, polymerase chain reaction (PCR), Northern blots, sequencing, reverse-transcriptase PCR, microarray technology, immunohistochemistry, enzyme-linked immunosorbent assay, flow cytometry mass spectrometry, Förster resonance energy transfer, time-resolved fluorescence energy transfer, amplified luminescent proximity homogeneous assay, fluorescence polarization, cell based assays or combinations thereof. In certain embodiments, the assay is a high throughput screening (HTS) assay.

As used herein, the term “contacting” (i.e., contacting a cell e.g. a cell, with a compound) includes incubating the compound and the cell together in vitro (e.g., adding the compound to cells in culture) as well as administering the compound to a subject such that the compound and cells of the subject are contacted in vivo.

As used herein, the term “test compound” refers to a compound that has not previously been identified as, or recognized to be, a modulator of the activity being tested. The term “library of test compounds” refers to a panel comprising a multiplicity of test compounds.

As used herein, the term “indicator composition” refers to a composition that includes a protein of interest (e.g., a coronavirus S1/S2 prefusion spike peptide), for example, a cell that has been engineered to express the protein by introducing one or more of expression vectors encoding the protein(s) into the cell, or a cell free composition that contains the protein(s) (e.g., purified naturally-occurring protein or recombinantly-engineered protein(s)).

As used herein, the term “cell” includes prokaryotic and eukaryotic cells. In one embodiment, a cell of the disclosure is a bacterial cell. In another embodiment, a cell of the disclosure is a fungal cell, such as a yeast cell. In another embodiment, a cell of the disclosure is a vertebrate cell, e.g., an avian or mammalian cell. In a preferred embodiment, a cell of the disclosure is a murine or human cell. As used herein, the term “engineered” (as in an engineered cell) refers to a cell into which a nucleic acid molecule e.g., encoding a coronavirus S1/S2 prefusion spike peptide.

As used herein, the term “cell free composition” refers to an isolated composition, which does not contain intact cells. Examples of cell free compositions include cell extracts and compositions containing isolated proteins.

The ability of the test compound to modulate a coronavirus S1/S2 prefusion spike peptide binding to the cognate receptor ACE2 can also be determined. Determining the ability of the test compound to modulate peptide binding to a receptor can be accomplished, for example, by coupling the cognate receptor ACE2 with a radioisotope or enzymatic label such that binding of ACE2 to a coronavirus S1/S2 prefusion spike peptide can be determined by detecting the labeled ACE2 in a complex. Alternatively, a coronavirus S1/S2 prefusion spike peptide could be coupled to a radioisotope or enzymatic label to monitor the ability of a test compound to modulate a coronavirus S1/S2 prefusion spike peptide binding to the cognate receptor ACE2 in a complex. Determining the ability of the test compound to bind to a coronavirus S1/S2 prefusion spike peptide or ACE2 can be accomplished, for example, by coupling the compound with a radioisotope or enzymatic label such that binding of the compound to a coronavirus S1/S2 prefusion spike peptide or ACE2 can be determined by detecting the labeled compound in a complex, e.g. an output value. For example, targets can be labeled with ¹²⁵I, ³⁵S, ¹⁴C, or ³H, either directly or indirectly, and the radioisotope detected by direct counting of radioemmission or by scintillation counting. Alternatively, compounds can be labeled, e.g., with, for example, horseradish peroxidase, alkaline phosphatase, or luciferase, and the enzymatic label detected by determination of conversion of an appropriate substrate to product.

It is also within the scope of this disclosure to determine the ability of a compound to interact with a coronavirus S1/S2 prefusion spike peptide or ACE2 without the labeling of any of the interactants, e.g. crystallography.

In certain aspects, the screening assays are cell-based assays. The cells used in the instant assays can be eukaryotic or prokaryotic in origin. For example, in one embodiment, the cell is a bacterial cell. In another embodiment, the cell is a fungal cell, e.g., a yeast cell. In another embodiment, the cell is a vertebrate cell, e.g., an avian or a mammalian cell. In a preferred embodiment, the cell is a human cell. The cells of the disclosure can express endogenous a coronavirus S1/S2 prefusion spike peptide or ACE2 or can be engineered to do so. For example, a cell that has been engineered to express the a coronavirus S1/S2 prefusion spike peptide and/or ACE2 can be produced by introducing into the cell an expression vector encoding the protein.

In another embodiment, the indicator composition is a cell free composition. a coronavirus S1/S2 prefusion spike peptide or ACE2 expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment, the amount of binding of a coronavirus S1/S2 prefusion spike peptide to ACE2 in the presence of the test compound is less than the amount of binding of a coronavirus S1/S2 prefusion spike peptide to ACE2 in the absence of the test compound, in which case the test compound is identified as a compound that inhibits binding of a coronavirus S1/S2 prefusion spike peptide to ACE2.

Binding of the test compound to a coronavirus S1/S2 prefusion spike peptide or ACE2 can be determined either directly or indirectly as described above. Determining the ability of a coronavirus S1/S2 prefusion spike peptide or ACE2 protein to bind to a test compound can also be accomplished using a technology such as real-time Biomolecular Interaction Analysis (BIA) (Sjolander, S., et al. 1991. Anal. Chem. 63, 2338-2345; Szabo, A., et al. 1995. Curr. Opin. Struct. Biol. 5, 99-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In the methods of the disclosure for identifying test compounds that modulate an interaction between a coronavirus S1/S2 prefusion spike peptide or ACE2, the complete a coronavirus S1/S2 prefusion spike peptide or ACE2 protein can be used in the method, or, alternatively, only portions of the protein can be used. In one embodiment of the above assay methods of the present disclosure, it may be desirable to immobilize either a coronavirus S1/S2 prefusion spike peptide or ACE2 for example, to facilitate separation of complexed from uncomplexed forms of one or both of the proteins, or to accommodate automation of the assay.

Binding of a test compound to a coronavirus S1/S2 prefusion spike peptide or ACE2 in the presence and absence of a test compound, can be accomplished in any vessel suitable for containing the reactants. Examples of such vessels include microtiter plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or a coronavirus S1/S2 prefusion spike peptide or ACE2 protein, and the mixture incubated under conditions conducive to complex formation (e.g., at physiological conditions for salt and pH). Following incubation, the beads or microtiter plate wells are washed to remove any unbound components, the matrix is immobilized in the case of beads, and complex formation is determined either directly or indirectly, for example, as described above. Alternatively, the complexes can be dissociated from the matrix, and the level of binding or activity determined using standard techniques.

Other techniques for immobilizing proteins on matrices can also be used in the screening assays of the disclosure. For example, either a coronavirus S1/S2 prefusion spike peptide or ACE2 can be immobilized utilizing conjugation of biotin and streptavidin. Biotinylated protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with protein but which do not interfere with binding of the proteins can be derivatized to the wells of the plate, and unbound a coronavirus S1/S2 prefusion spike peptide or ACE2 protein is trapped in the wells by antibody conjugation. Methods for detecting such complexes, in addition to those described above for the GST-immobilized complexes, include immunodetection of complexes using antibodies reactive with XBP-1 or HIF1α, as well as enzyme-linked assays which rely on detecting an enzymatic activity associated with a coronavirus S1/S2 prefusion spike peptide or ACE2.

Another aspect of the disclosure pertains to kits for carrying out the screening assays, modulatory methods or diagnostic assays of the disclosure.

### EXAMPLES

### Example 1: Methods

The following methods were used in Example 2:

Building the Spike Model with all RBDs Closed.

The model described here, of the SARS-Cov-2 spike protein ranged from residue Q14 to V1164, covering most of the parts of S1 and S2. To avoid the influence of charged termini, three acetyl capping groups were introduced to the 3 N-terminals of the 3 protomers. Model building started from the closed model for the glycosylated spike that was described previously (2), with additional changes described here in order to incorporate additional structural details that are resolved in newer cryo-EM structures. Steered MD (SMD) was used to change the structure to match the wild-type closed spike in cryo-EM structure 6XR8, which resolved the coordinates for the fusion peptide proximal region FPPR (1) under the CTD12 domain; this is missing in many other structures. The collective variable (CV) used for SMD was RMSD with all CA atoms in 6XR8. The RMSD was gradually reduced from the initial value of 5.2 Å to 0, using 100000 kcal/(mol Å²) harmonic restraint during 20 ns. After SMD, a few MD and SMD steps were applied to fix some glitches in the model introduced during SMD. Additional equilibration was conducted on the sidechains and glycans that were not included in the SMD restraint. While keeping the same CA RMSD restraint of 100000 kcal/(mol Å²), the system was heated from 310 K to 400 K during 2 ns, kept at 400 K for 16 ns and gradually cooled back to 310 K. The RMSD restraints were released and the system was again heated to 400K during 2 ns, then kept at 400K for 8 ns. A loop region not resolved in 6XR8 was observed to be distorted, so another CV was added on a loop region of S1 (residue A61 to F79) using the CA atoms of the same loop of protomer 2 from the first heating step as reference, and SMD was applied at 400 K over 20 ns while maintaining the RMSD restraint. A glycan branch (connected to N17 of protomer 1 in the model) was found to have become trapped between protein chains; it was removed with additional SMD on two atoms. The distance between the C1 atom of residue 689 of the model, which is in the glycan branch connected to N74 of protomer 1, and the CA of Glu339 of protomer 1 RBD was increased from 50.5 Å to 110 Å during 14 ns of SMD at 400 K. No RMSD based restraint was used in this step. The SMD restraint using 6XR8 as reference was added back. The total RMSD was reduced from 1.26 Å to 0.21 Å by gradually increasing the force constant from 1000 to 100000 kcal/(mol Å²) during 10 ns. The simulation temperature was gradually decreased during the first 8 ns from 400 K to 310 K, then kept at 310 K for the last 2 ns. Another glycan problem was fixed in the same way. Atoms C1 of residue 1997 of the numbering herein, which is in the glycan branch connected to N17 of protomer 2 of the model, and atom CA of C136 of protomer 2 of the model was selected for SMD, and their distance was increased from 12.2 Å to 30 Å during 4 ns at 310 K. Next, the chemical composition of the spike model was adjusted to better match 6XR8. Short sequences Ace-Asn14-Cys15 were added to the 3 N-termini of S1 to resolve a missing disulfide bond. Disulfide bonds were added between C15-C136 (in NTD) and C840-C851 (in FPPR). All O-linked glycans were deleted from the original model due to doubts (3, 4) about their presence in the trimer. The spike protein was solvated using 31 Å minimum distance from solute to box edge with 371793 water molecules added. A large box was used to ensure adequate solvation of the flexible glycans, and enclosure of the RBDs after opening. 373 sodium ions and 353 chloride ions were added. The same equilibration protocol was used as in previous work (2), except that in the first minimization step the default 10 steps of steepest descent algorithm were used followed by conjugate gradient. 6XM5 also resolved a loop region in CTD2 missing from our original model template. The CA atoms of V608 to G652 of chain A from 6XM5 were used as reference. The RMSD of three regions were reduced from their initial value (around 5-6 Å) to 0 using 50000 kcal/(mol Å²) restraint in 10 ns SMD. Finally, the FPPR structure solved in PDB 6XM5 (5) was used as an alternative FPPR conformation (hereafter referred as “Kwong FPPR”, with the one from 6XR8 referred as “Chen FPPR”). SMD was used to change the conformations. CA atoms of 5816 to D867 of chain B of 6XM5 were used as the structural reference. The RMSD of the three FPPRs was reduced from around 8 Å to 0 using 50000 kcal/(mol Å²) restraint during 10 ns SMD. Separate structures were built using both the Chen FPPR and Kwong FPPR; the Chen FPPR was employed for closed states (consistent with 6XR8), and the structure with Kwong FPPR was used for building the 3-up models since the Chen FPPR creates steric clash with the CTD1 location in 7CAK.

Building the Spike Model with all RBDs Open (3-Up)

The 3-up model was built up based on the cryo-EM model of the 3-up spike with each RBD bound to an H014 antibody Fab fragment in the 7CAK structure. The closed spike model was used as the starting model. All 3 RBDs were opened by SMD using our equilibrated 1-up model from previous work (2) as reference. The RMSD selection included CA atoms of residues 338-517, 324-327, 538-585, 747-782, 946-966 and 987-1034, which include the RBD, CTD1 and helical segments in UH, HR1 and CH. 3 RMSDs was reduced from ˜12.5 Å to 0 using 100,000 kcal/(mol Å²) restraint over 20 ns. After generating the 3-up model, 270 ns of regular MD was carried out. Since differences were noted in the experimental structures of the 1-up and 3-up spike, the 3-up structure after MD was steered to match 7CAK by reducing RMSD from 12.2 Å to 0 using 100,000 kcal/(mol Å²) force constant over 30 ns. All CA atoms resolved in 7CAK were used for steering. The RMSD restraint was maintained on the CA atoms for an additional 30 ns to relax the model before unrestrained MD runs. All simulations were run at 310 K.

Building the Model Systems with a Subset of the S2 Trimer

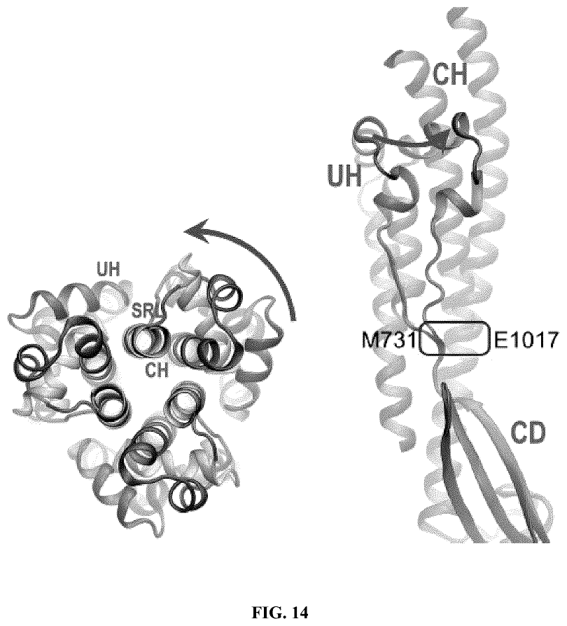

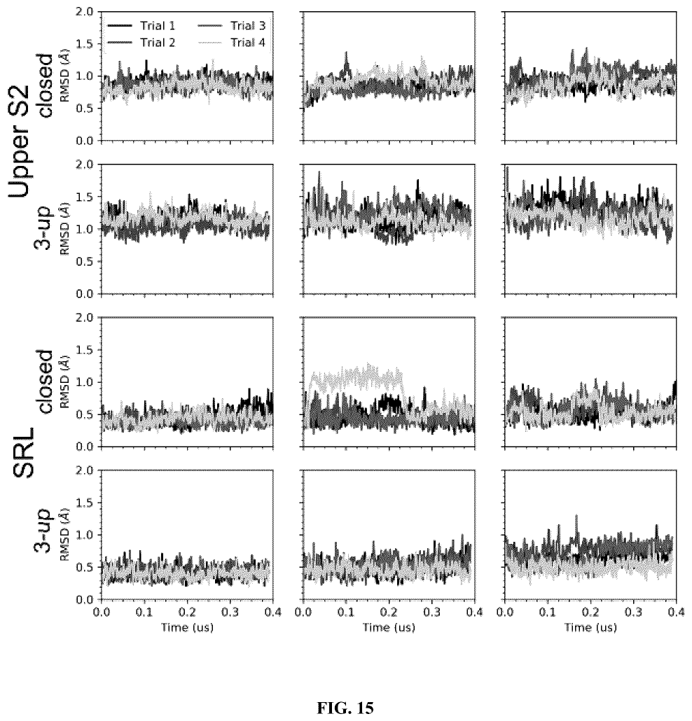

Two small model systems for the S2 subunit were built, both based on the wild-type pre-fusion 6XR8 structure. This approach is similar to the peptide fragment experiments used to determine the spring-loaded mechanism of influenza hemagglutinin (6, 7). All amino acids below the fulcrum of S2 rotation (FIG. 14) were removed. In the “medium” system, the central components of all three protomers in the trimer were retained, including the CH, SRL, HR1 and UH (M731-K776, D950-R1019) (FIG. 15). The N- and C-terminal ends of all six chains were capped with neutral termini and restrained to their initial coordinates during all MD steps described below. In the “small” model, the medium system was further truncated by deleting the HR1 chains from D950 to S975, with neutral capping added but no restraints applied to V976. Both systems were simulated in explicit water.

The small model system was initially simulated in TIP3P 3-point water model for speed, since our goal was to carry out a long unrestrained simulation to test for helix extension. The ff14SB force field that is optimized for TIP3P was used. Water was added with a minimum distance of 8 Å from solute to the box edge, resulting in addition of 6054 water molecules. The system was equilibrated in a series of stages including 1000 steps of minimization, 100 ps of heating from 100 K to 298K at constant volume and with positional restraints of 10 kcal/mol/Å² on all non-solvent atoms; 200 ps at constant pressure to equilibrate density with positional restraints of 10 kcal/mol/A2 on non-solvent atoms; 200 ps with positional restraints of 10 kcal/mol/A2 on the capping groups at the base of the fragment (see above). This was followed by 1.65 microseconds of MD at 350K and 1 bar, maintaining the restraints on the fragment caps. The increased temperature was employed based on experiments (6) that show elevated temperature leads to activation of hemagglutinin at neutral pH. Refinement was carried out for the final structure by converting to the more accurate but slower ff19SB force field with OPC 4-point explicit water. The structure was equilibrated using the same protocol as described above, followed by 1 microsecond of MD at 310K with restraints only on the lower fragment caps.

The medium model system simulations aimed to create a model for the unfolded SRL and location of the new, longer CH α-helix. The ff19SB force field was retained with the OPC water model. The system was equilibrated using the same protocol as the small model system. Following 400 ns of MD at 330K and 1 bar, the final snapshot was used to initiate SMD simulations to extend the central helices. The medium model was steered to unfold the SRL and extend the CH cap by using the uncapped small model as reference. A separate CV was applied to each protomer. The RMSD region included CA atoms of N978 to E1017 (including the short upper HR1 helix, continuing down CH). N978 was chosen as the end of the restrained region since the simplest hypothesis for extension of CH was that the upper 2-turn α-helix of HR1 (L977-L984) would add to CH, and that the SRL (S₉₆₇-V966) would remain non-helical.

As with the small model, the free ends of the protein chains at the base of the model were restrained to their initial positions using 10 kcal/(mol Å²) force constant. RMSDs for the CV regions were reduced from around 6 Å to 0 using 10000 kcal/(mol Å²) restraint during 40 ns at 330 K. Following SMD, the structure was relaxed for 110 ns at 330 K to optimize the conformation of the SRL linking the extended CH and the lower HR1. Distance restraints were applied to maintain hydrogen bonds on the newly extended helix, and positional restraints on the fragment base were maintained. Next, a simulation of 887 ns was carried out for the same system at 300 K and 1 bar, again using FF19SB and OPC, with restraints only on the fragment base. The final snapshot was used as an SMD target for the full spike system.

Building the 3-Up Spike with Partially Extended CH and Unfolded SRL

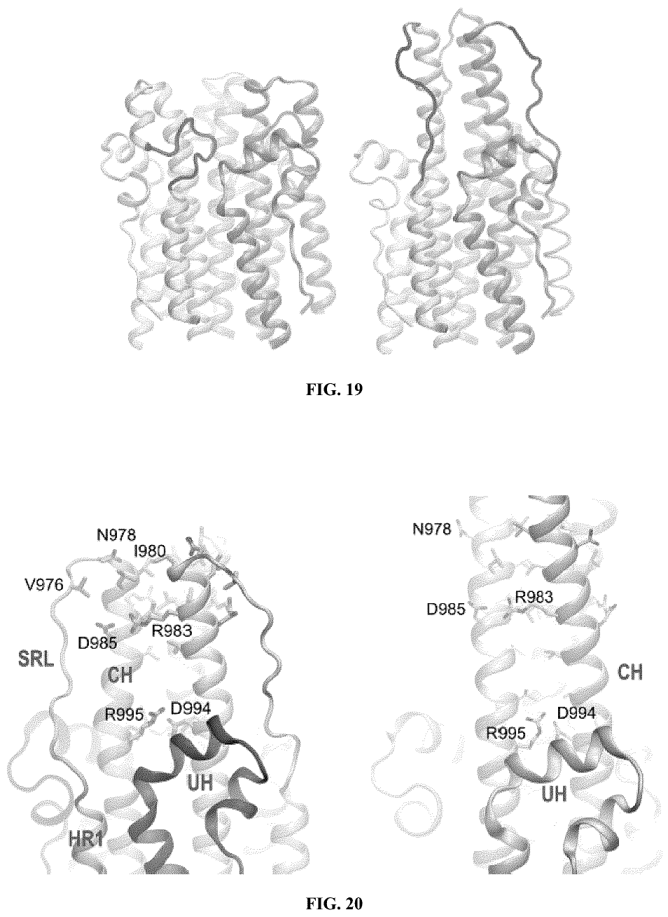

The model building started from the MD snapshot of the 3-up model after 210 ns unrestrained MD. The final structure of the medium model with all 3 CH extended (FIG. 19) was used as a structural reference. The RMSD region included the CA atoms of residues K964 to Q1010 of all three protomers. The backbone atoms (CA, C, N, O) of the rest of the system (Q14-R685, S686-L962, L1012-V1164) were restrained using 10.0 kcal/(mol Å²) positional restraint. The RMSD was reduced from 9.4 Å to 0 using 100000 kcal/(mol Å²) restraint during 30 ns of SMD at 310 K. After SMD, the system was relaxed during 30 ns MD at 310 K while applying 0.1 kcal/(mol Å²) positional restraints to all CA atoms.

Nudged Elastic Band Optimization of the CH Extension Pathway in the 3-Up Spike

The partial nudged elastic band (NEB) method (8) was used as implemented (9) on GPUs in Amber to generate a minimum energy path for the CH extension process. The 3-up spike structure from MD initiated from 7CAK and 3-up spike with extended central helices (see above) served as the two endpoints, which remain static throughout the calculation. Thirty additional beads were simulated in parallel between these two endpoints, with the first 16 initiated from the first endpoint and the last 16 from the second endpoint. The NEB springs were applied with a 1 kcal/(mol*Å²) force constant to the N, CA and C atoms of the entire protein, as well as the carbon atoms of the glycans attached to N234 since previous work (10) implicated this glycan site in RBD dynamics. NEB was run in a four-step protocol that involved heating, equilibrating, annealing, and MD. The set of 32 images were first heated from 100 to 300 K over 0.5 ns at constant volume, followed by 1 ns of equilibration at constant pressure of 1 bar and temperature of 300K. The system was then annealed by heating to 400 K over 2 ns, maintaining 400 K for 1 ns, cooling to 300 K over 2 ns, followed by a final 10 ns at 300 K to generate the final path. Following MD, a further 240 ns of fully unrestrained MD was performed for the 3-up spike with extended CH.

Energy Postprocessing of Simulation Snapshots

The solvation free energy of R1000 was estimated using Poisson-Boltzmann calculations, with a similar strategy as reported (11) previously. Only the partial charges of the three R1000 were kept (including backbone and sidechains), while the partial charges of all other atoms were set to zero. The Amber PBSA module was used for PB calculations on the full spike. Default options were used except a grid size of 1 Å to reduce the memory requirements. Mbondi (12) atomic radii were used. Dielectric constants used in PB calculation were 78.5 and 1 for solvent and solute, respectively. Snapshots spaced 10 ns apart were taken from 250 ns of each simulation, resulting in 25 frames each for closed, 3-up and 3-up extended simulations, respectively.

Calculation of the UH Rotation Angle

Four Center of Mass (CoM) points were defined to measure a dihedral that quantifies the UH rotation for each protomer. Amino acids N1054, S1055, G1060, and V1061 in the connector domain were used to calculate the first CoM point. The second point included T778, N779, E780, V781 (UH), the third point included R765 to T768 (UH) and the fourth point included T747 to 5750 (UH). Only the Ca atoms were used to calculate the CoM locations. The dihedral values were calculated using cpptraj (13).

Analysis of Structures from the PDB

81 structures were extracted from the PDB (Table 1). Only SARS-CoV-2 spike trimers including both the 51 and S2 subunits were included, except for the post-fusion 6XRA which includes only the S2 subunit. No model building was performed. If any atoms were missing from a structure, it was not included in that particular analysis. Chain information was corrected in some structures such that protomers were counterclockwise as chains A, B and C. PDB files with amino acid position numbering that did not correspond to the Wuhan sequence (e.g., 986/987 at the CH top) were excluded.

The RBD to HR1 distance was defined as the distance from the N atom of 5383 to the O atom of R983. These amino acids have been replaced successfully with a disulfide bond (14-16), substantiating their close contact. The RBD to CH distance was defined as the distance between CA atoms of D427 and K986. Using the RBD-HR1 distance, 159 protomers exhibited a closed RBD, and 78 protomers exhibited an open RBD. Average values are reported, with uncertainties reflecting the standard deviation of the distribution.

Salt bridge distances were calculated as the minimum value of six distances calculated between the two carboxyl oxygen atoms of Asp or Glu side chains and the three nitrogen atoms in the guanidino group of Arg. Asp to Ser distances were calculated as the minimum distance from either carboxyl oxygen to the hydroxyl oxygen of Ser. R1000 to S975 or 1742 hydrogen bonding was calculated as the minimum distance of the three guanidino nitrogen atoms to the backbone oxygen of S975/I742. Uncertainties quoted on data from this structure set indicates the standard deviation over the measurement in all of the PDB structures.

Water Density Analysis

The grid command of cpptraj (13) was used to analyze the water density around R1000. 4200 frames from 420 ns of MD simulation of the closed model were taken for analysis. The structures were first aligned by overlapping residue V963-S1003 of the first protomer. Then the oxygen atoms of water molecules within a cubic region which centered at CE1 atom of F970 of the first protomer with edge length of 8 Å were counted using a bin width of 0.5 Å. The grid was visualized in VMD (17).

Miscellaneous All structure images in this work were made using VMD (17) version 1.9.5a4. Structural analysis of the PDB structures and MD simulations was performed with cpptraj (13).

### Example 2: Receptor Binding May Directly Activate the Fusion Machinery in Coronavirus Spike Glycoproteins

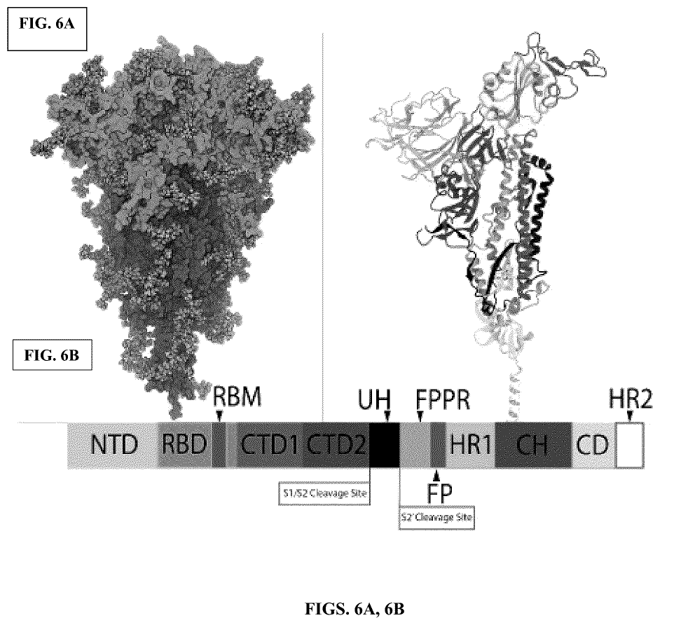

A coronavirus spike is a homotrimeric glycoprotein, with each protomer consisting of two subunits 51 and S2; both are heavily decorated with glycans (1). The N-terminal 51 subunits sit atop the spike and are responsible for recognizing and binding a host cell receptor and stabilizing the S2 core (2-9). In SARS-CoV-2, each 51 subunit consists of an N-terminal domain (NTD), a receptor binding domain (RBD), and two C-terminal domains (CTD1 and CTD2); the S1/S2 interface lies at the C-terminal end of CTD2 (FIGS. 6A, 6B) (10-12). The RBD in the 51 subunit is responsible for recognizing and binding angiotensin converting enzyme 2 (ACE2) (FIGS. 1A, 1B) (3, 6-9, 13). The RBD alternates between at least two main conformational states relative to the remainder of the spike: ‘open’ and ‘closed’ (11, 12, 14, 15). An open RBD is a prerequisite for ACE2 binding; in the closed state binding of ACE2 is precluded by a steric clash with the RBDs of other protomers (12, 14, 16, 17).

The fusion machinery of the spike is contained in the S2 subunit (18). The S2 is composed of an upstream helix (UH) at the N-terminus, the fusion peptide proximal region (FPPR), the fusion peptide (FP), the first conserved heptad repeat region (HR1), a long central α-helix (CH), and a connector domain (CD) that leads into the C-terminal ‘stalk’ which contains the second heptad repeat (HR2), and the transmembrane domain (TM) that spans the viral membrane, followed finally by an intracellular region (FIGS. 6A, 6B) (11, 12, 19).

The SARS-CoV-2 spike possesses two distinct cleavage sites that must both be processed by host proteases to foster efficient membrane fusion (S1/S2 and S2′) (4, 5, 20-22). The priming site at the S1/S2 boundary disconnects the S1 and S2 subunits, permitting eventual S1 dissociation (shedding). Cleavage at the S1/S2 site in SARS-CoV-2 is thought to occur during the secretory pathway; this is possible due to insertion of a dibasic furin recognition sequence (₆₈₂RRAR₆₈₅) that is not present in SARS and many other coronaviruses (21). The activation site S2′ (₈₁₄KRSF₈₁₇) (20, 23) is in the S2 domain at the FPPR/FP boundary, and is believed to be cleaved by membrane-bound host proteases such as TMPRSS2 (23-26) or cathepsin (23, 27).

At some point after the S1 region binds ACE2 (FIG. 1B), the S1 subunits dissociate to expose the metastable S2 core (FIG. 1C), which is “spring-loaded” similar to influenza hemagglutinin (28). S2 subsequently undergoes large-scale refolding that initiates membrane fusion (18, 19, 29-31). S2′ cleavage releases the fusion peptides at the end of HR1, although the timing of cleavage relative to ACE2 binding and S1 shedding remains unclear. The FP diffuse to and insert into the host cell membrane, presumably forming a short-lived pre-hairpin intermediate tethering the two membranes (FIG. 1D) (18, 29, 32). This structure refolds again to adopt the post-fusion structure, with a six-helix bundle (23, 29, 33) formed from the HR1 and HR2 helices that are attached to the spike stalk and fusion peptides, respectively (FIG. 1E). These dramatic rearrangements allow one or more spikes to bring the two lipid bilayers into contact, generating a fusion pore and releasing the virion's contents into the host cell (29).

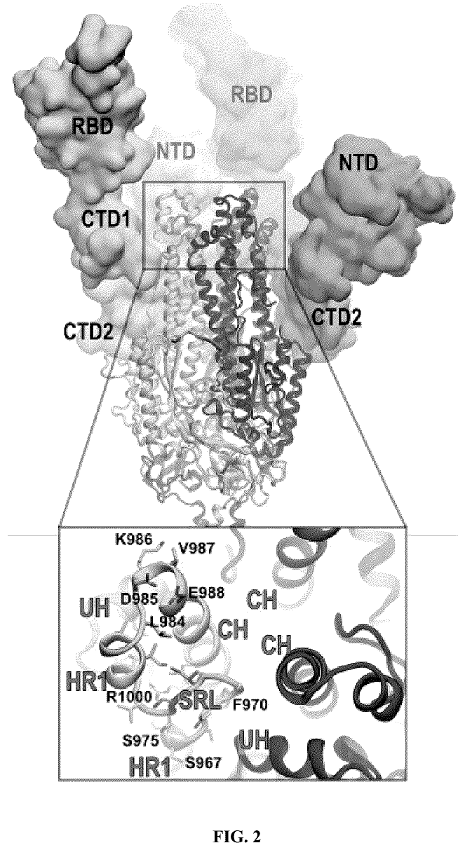

What couples the receptor-binding function of the S1 subunit to the membrane fusion machinery of the S2 subunit? The “ratcheting” mechanism (34) suggests that opening of multiple RBDs leads to significant loss of S1-S2 contact area, leading to S1 shedding, after which the metastable S2 trimer refolds (29). However, experiments have imaged the pre-fusion spike bound to 1, 2 or 3 ACE2 proteins (FIG. 2), suggesting that RBD ratcheting is insufficient to induce S1 shedding, even with S1/S2 cleavage (6). It is plausible that cleavage at S2′ is a prerequisite for 51 shedding, but the S2′ recognition motif appears inaccessible to proteases in pre-fusion spike structures. Other host factors may participate in spike-mediated viral entry (35-37), but in vitro S1 shedding and adoption of post-fusion structure can occur in their absence (19; 29).

One possible explanation for these observations is that all available experimental structures with more than one open RBD employed sequences with stabilizing amino acid substitutions at the N-terminal of the CR α-helix (such as the widely employed “2P” spike (39), K986P/V987P). The enhanced stability of the 2P spike has led to many valuable studies of spike structure and function, as well as CON/ID-19 vaccines. The 2P modification is intended to block CH extension, since proline is relatively rigid and lacks the ability to donate backbone hydrogen bonds needed to extend the CH α-helix (39).

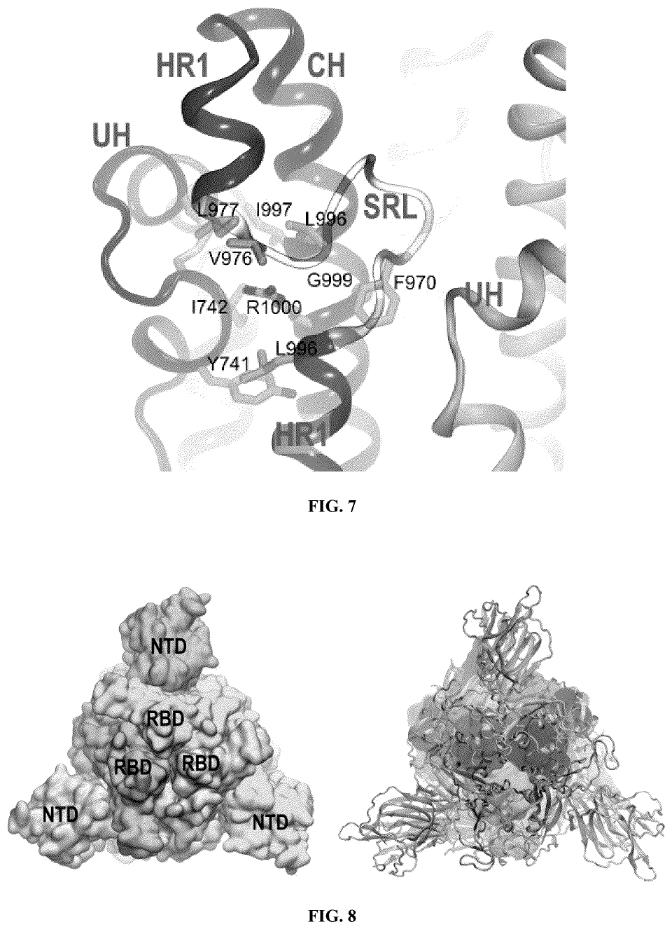

Herein, a model is described for how receptor binding may trigger significant conformational changes in the S2 fusion machinery prior to S1 shedding. In this model, lengthening of the central coiled-coil occurs prior to S1 shedding; this shares features with a reported intermediate state in influenza hemagglutinin, in which α-helix extension in HA2 occurs prior to separation from HA1 (6). An important role is played by an unusual loop in the S2 subunit, referred to herein as the serine-rich loop (SRL) since four of ten amino acids are serine (S₉₆₇SNFGAISSV₉₇₆). The SRL is located between the 3rd and 4th α-helices of HR1, near the location of the 2P substitution at the top of the CH (FIGS. 2, 7). In addition to providing a hypothesis for events immediately following spike-receptor binding, the model also suggests caution when interpreting data obtained with stabilizing substitutions in the context of the spike-mediated fusion mechanism.

Results

Analysis of Experimental Structures of the SARS-CoV-2 Spike from the PDB.

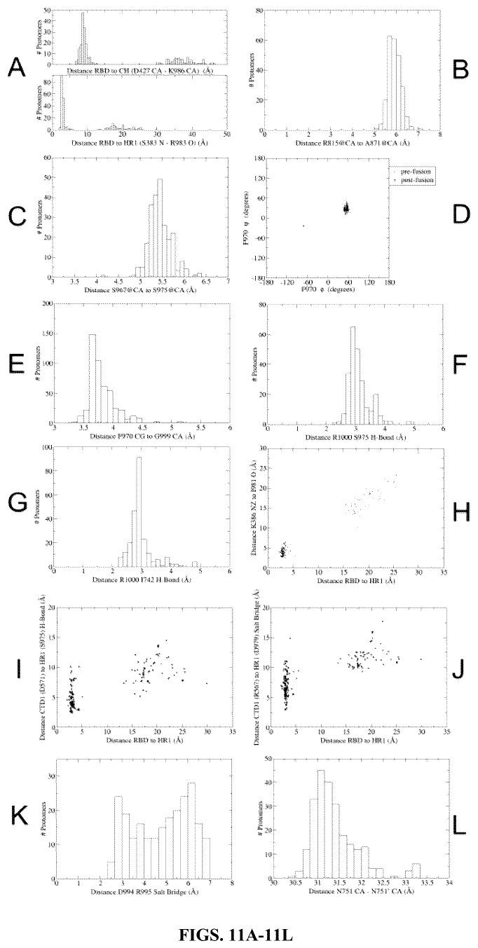

Representative experimental structures in four different states: 6XR8, 6VSB, 7CAK, and 6XRA were selected. Although most structures include the 2P stabilizing substitutions (39), a few studies did not (referred to here as “wild-type”). These include the 6XR8 (19) pre-fusion structure with all three RBDs closed, and the 6XRA (19) post-fusion structure. Also included, were the 6VSB (11) 2P-spike with 1 RBD open (“1-up”), and the 7CAK(38) 2P-spike with all 3 RBDs open (“3-up”) and bound to antibody Fab proteins (7CAK has high similarity to the 3-up 7A98 (6) structure with each RBD bound to an ACE2 domain). Due to limited resolution in cryo-EM models, analysis and visualization of these four structures is supplemented with data extracted from 81 SARS-CoV-2 spike structures obtained from the PDB (80 pre-fusion and 1 post-fusion, data in FIGS. 11A-11L, with PDB codes in Table 1). Histograms of distances between the RBD and the top of CH or HR1 are shown in FIG. 11A. In all structures, R815 at the S2′ cleavage site appears inaccessible to proteases and closely packed against A871, with α-carbon distances of 5.9±0.3 Å in the 80 pre-fusion spike structures (FIG. 11B).

TABLE 1
PDB codes for the 81 experimental spike structures used in data analysis. All structures are pre-fusion except 6XRA.

| | | | | | | | | | | | | |
|---|---|---|---|---|---|---|---|---|---|---|---|---|
| 6VSB | 6VXX | 6WPS | 6WPT | 6X29 | 6X2A | 6X2B | 6X2C | 6X6P | 6XCM | 6XCN | 6XEY | 6XF5 |
| 6XKL | 6XLU | 6XM0 | 6XM3 | 6XM4 | 6XM5 | 6XR8 | 6XRA | 6Z43 | 6Z97 | 6ZDH | 6ZGE | 6ZGG |
| 6ZGH | 6ZGI | 6ZOW | 6ZP5 | 6ZP7 | 6ZWV | 6ZXN | 7A29 | 7A4N | 7A93 | 7A94 | 7A95 | 7A96 |
| 7A97 | 7A98 | 7AD1 | 7BYR | 7C2L | 7CAI | 7CAK | 7CHH | 7CN9 | 7JJI | 7JV4 | 7JV6 | 7JVC |
| 7JW0 | 7JWB | 7JZL | 7JZN | 7K43 | 7K4N | 7K8S | 7K8T | 7K8U | 7K8V | 7K8W | 7K8X | 7K8Y |
| 7K8Z | 7K90 | 7KDG | 7KDH | 7KDI | 7KDJ | 7KDK | 7KDL | 7KE4 | 7KE6 | 7KE7 | 7KE8 | 7KE9 |
| 7KEA | 7KEB | 7KEC | | | | | | | | | | |

FIG. 2 shows the 3-up spike, where the open RBDs expose the top of the S2 subunit trimer. This region contains the three central helices (CH) at the core, with each protomer forming a helix-turn-helix motif (HTH) connecting HR1 to CH. The top of the wild-type CH exhibits features of an N-capping motif (40), with the D985 side chain hydrogen bonding to the backbone N of E988 and hydrophobic contacts stabilizing a turn that leads from HR1 into the CH (FIG. 2). The HTH are surrounded by the three upstream helices (UH); the upper horizontal α-helix of each UH forms a close contact with the CH of the same protomer.

An unusual loop is present near the top of S2. Moving from the HTH motif down the spike along HR1, two of the four α-helical segments of the pre-fusion HR1 (helix-3 and helix-4) are interrupted by the serine-rich loop. The SRL adopts a “C” shape, ending in nearly the same location as it started (FIG. 2), with the α-carbon atoms of S₉₆₇ and S975 separated by 5.4±0.2 Å in the PDB structure set (FIG. 11C).

The pre-fusion SRL has several unusual features. The segment from N969 to A972 forms a type-I′ β-turn (turn type ad (41)), where amino acids in the a and d positions both adopt positive φ backbone dihedral values. G971 occupies the d position, but the a position is occupied by the conserved F970, which likely is strained due to this disallowed backbone conformation (adopted in all pre-fusion structures but not in the post-fusion state, FIG. 11D). The side chain of F970 is packed tightly against G999 in the CH (FIG. 2, with a distance of 3.8±0.2 Å, FIG. 11E). Glycine is uncommon inside helices due to the additional entropic penalty of adopting a defined conformation, but any side chain at this position would clash with the F970 ring.

Another presumably strained feature of the pre-fusion SRL is the placement of R1000 inside a cluster of hydrophobic side chains capped by the SRL (FIGS. 2, 7). Arginine has a pKa near 14, and almost certainly remains charged even when buried in a nonpolar environment (42-44). The buried guanidino group is able to form only two hydrogen bonds (one to each side of the pocket), with backbone O atoms of 1742 on the UH and 5975 on the SRL (PDB data in FIGS. 11F, 11G). Cation burial may serve as a reservoir to store spike folding energy for release during the transition to the solvent-exposed environment observed in the post-fusion spike.

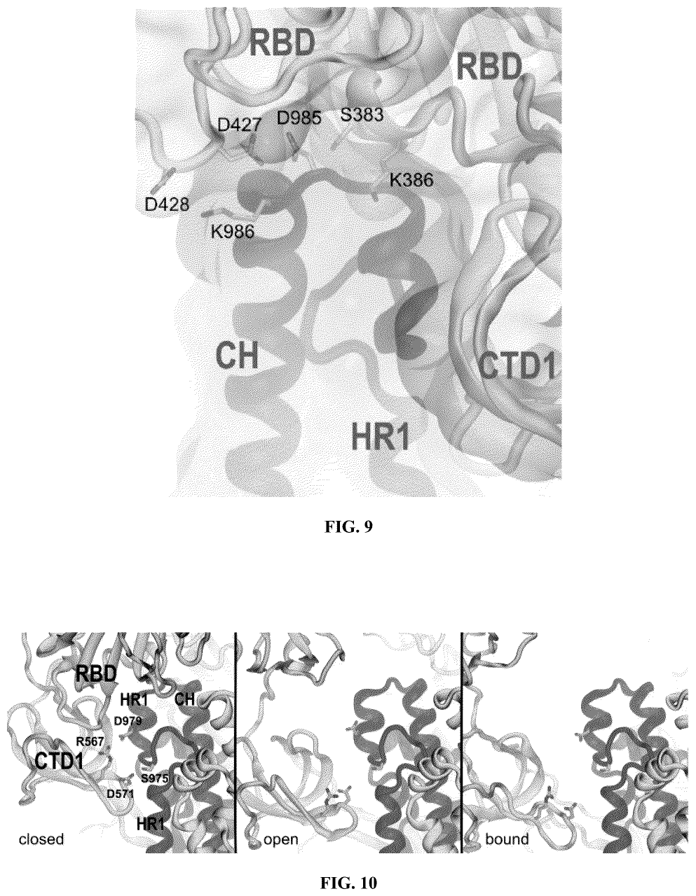

In the closed spike, each HR1-CH turn is covered by the RBDs of both other protomers (FIG. 8), with the counterclockwise protomer contacting the CH and clockwise protomer contacting HR1. Specific interactions are shown in FIG. 9. D428 is in the proximity of CH K986 (P986 in the 2P spike), potentially forming a favorable interaction that stabilizes the closed RBD (19). The RBD of the counterclockwise protomer forms extensive interactions with the top of HR1 and the turn connecting it to CH, including a hydrogen bond between the 5383 sidechain OG and the backbone of D985, and the backbone N of S383 with the backbone O of R983 (PDB data in FIG. 11A). The side chain amine of K386 on nearly every closed RBD forms a C-capping interaction with HR1, stabilizing the exposed backbone O of 1981 (FIG. 11H). Overall, these contacts with the two RBDs help stabilize the ends of both α-helices in the HTH.

RBD opening results in extensive loss of contacts between the S1 and S2 subunits. Our representative systems for the open spike include the 1-up 6VSB, and 3-up 7CAK (FIG. 2); both include the stabilizing 2P substitution. As expected, the contacts between the RBD and the S2 HTH motif are lost when the RBD opens (FIG. 2). However, additional S1-S2 interactions lower down the spike are also disrupted. As the RBD opens, the attached CTD1 domain moves outward (6), though to a lesser extent than the RBD. This shift results in loss of contacts between side chains on the CTD1 and the short upper helix of HR1, including R567-D979 and D571-S975. The outward shift of CTD1 leaves this HR1 segment completely free of contact with the S1 subunit, even more so when the RBD is engaged by ACE2 or antibodies (FIG. 10). Data extracted from the 80 pre-fusion structures support a strong correlation between RBD opening and loss of CTD1-HR1 contacts (FIGS. 11I-11J).

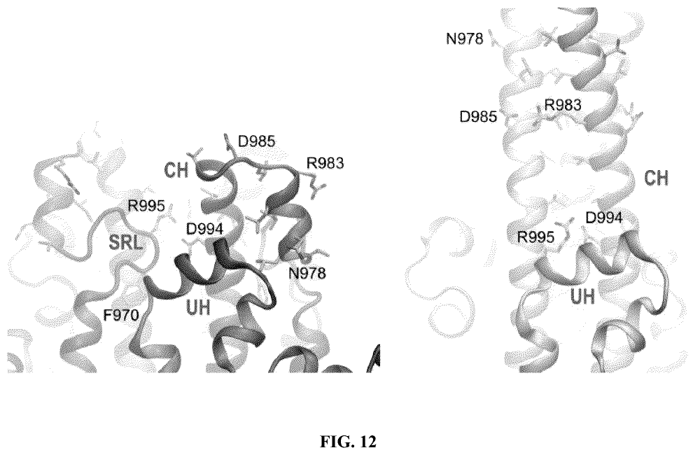

Comparing the pre-fusion and post-fusion spike reveals important changes in the region near the SRL. In the post-fusion state, the S1 subunit has dissociated and only the S2 subunit is present (FIG. 1E). The most obvious conformational change is the dramatic extension of CH, in which the HTH motif, SRL and the remainder of HR1 rotate upward to form a single continuous helix with CH, which adopt the coiled-coil motif that extends the FP toward the host cell membrane (19, 29). The process may be driven in part by energetically favorable changes; compared to the pre-fusion spike, R1000 becomes solvent-exposed, F970 adopts an allowed backbone conformation (FIG. 11D), hydrophobic side chains from the HR1/SRL form a tightly packed inter-protomer hydrophobic cluster, and six new inter-protomer salt bridges are formed within a stretch of three turns of α-helix (FIG. 12).

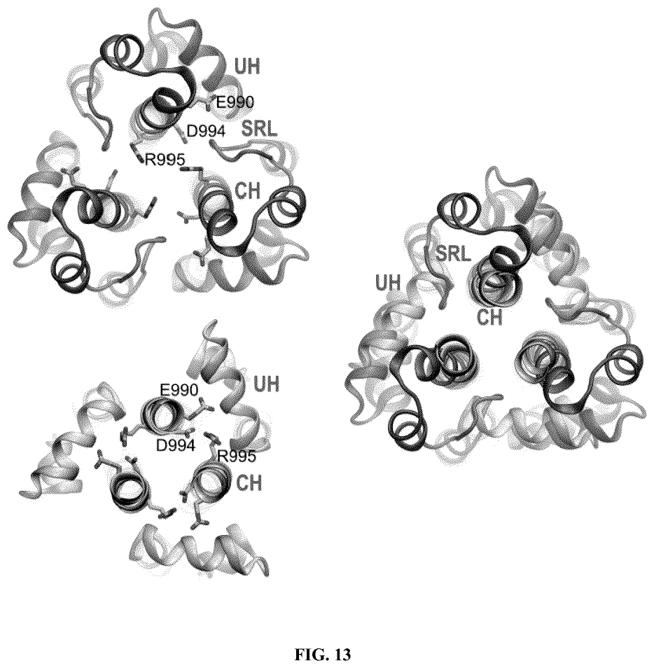

Three of these salt bridge pairs (D994-R995′) are close to the site of the SRL in the pre-fusion structure. The SRL is deeply inserted between the UH and CH of neighboring protomers, and the SRL appears to sterically block R995 from approaching D994 (FIG. 13; with a broad distribution of D994-R995′ distances in PDB structures, FIG. 11K). Relocation of the SRL in the post-fusion structure is associated with closer approach of the UH and CH, permitting formation of the D994-R995′ salt bridges (FIG. 13). The other three new inter-protomer salt bridge pairs in the post-fusion structure (R983-D985′) involve amino acids from the upper short helix-4 of HR1 which are distant in the pre-fusion structure but become neighbors in the coiled-coil of the post-fusion state (FIG. 12).

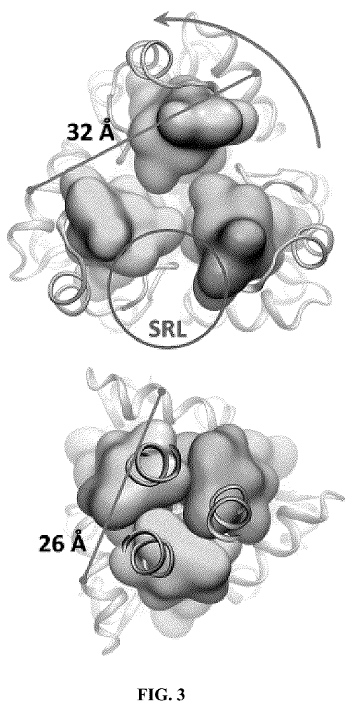

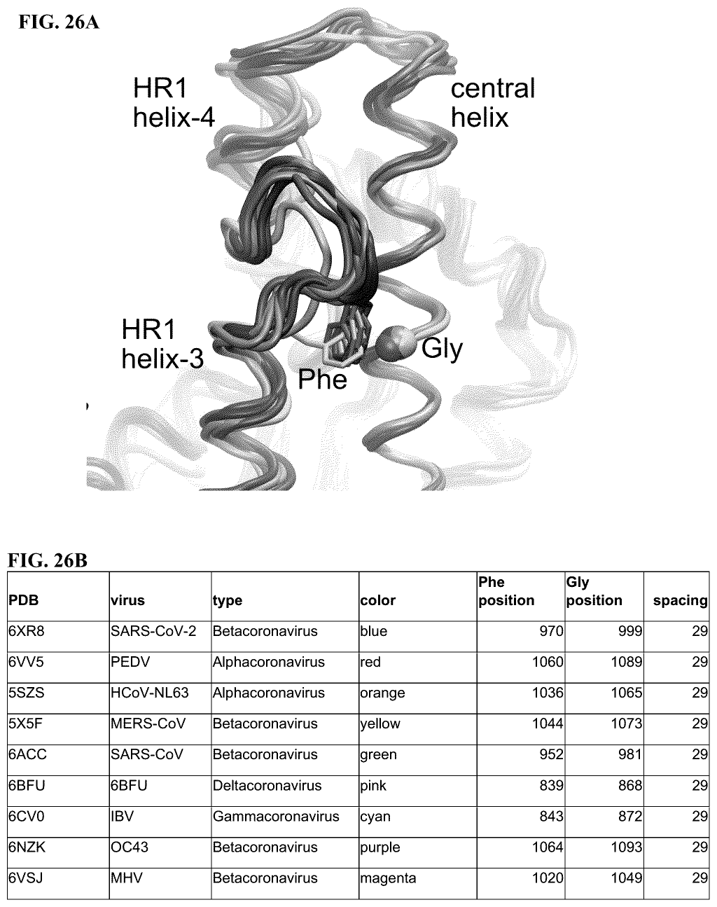

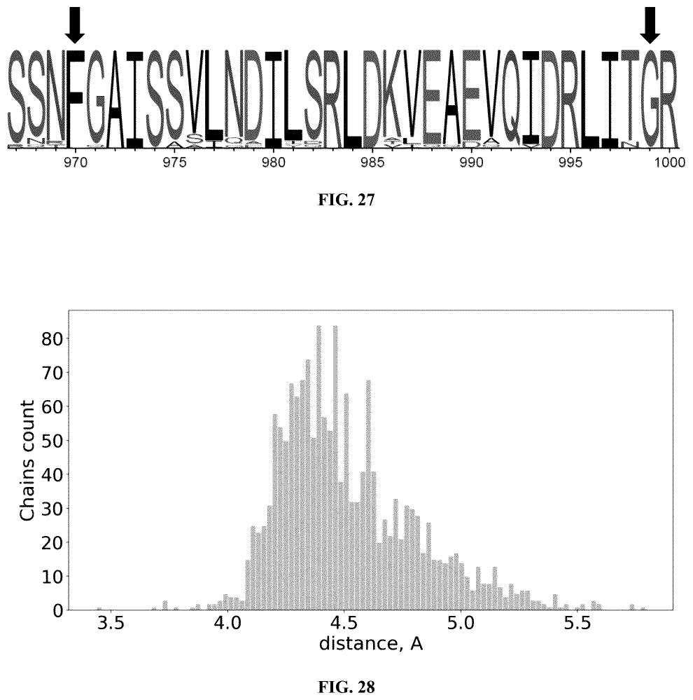

The upper S2 rotates relative to the lower S2 during membrane fusion (45). Despite the dramatic differences between the pre-fusion and post-fusion structures, the S2 structure near the base of the CH shows minimal changes. Overlap on this region highlights a significant twist in S2 that accompanies the transition, with the upper CH and UH rotating about a fulcrum that is highly localized near M731 and E1017 (FIG. 14). The net result is that the post-fusion S2 becomes smaller and more tightly packed than in the pre-fusion spike; these changes may be blocked until the SRL wedge is removed from between CH and UH (FIG. 3). The distance between N751 α-carbon pairs is reduced from 31.4±0.5 Å in the PDB pre-fusion structures (FIG. 11I) to 26 Å in the post-fusion spike.

A proposed role for the SRL in triggering of spike-mediated membrane fusion. Unless the SRL serves a functional purpose, one would expect these strained amino acids (e.g., F970, G999, R1000) to be replaced during viral evolution. What mechanistic role could necessitate their conservation? When the RBDs open, the packing around the upper helix-4 of HR1 is released, while the HR1 helix-3 below the SRL remains constrained by the S1 CTD1 (FIG. 10).

It is proposed herein that the RBD opening may provide the space needed to rotate upward the short HR1 helix-4, extending the CH by 2 turns. The N-terminal of the lengthened helix is able to remain connected to the more-distant HR1 helix-3 through unfolding of the compact SRL. SRL unfolding could be compensated energetically by relaxation of F970, solvent exposure of R1000, and formation of the D994-R995′ salt bridges, while adding the HR1 helix-4 to the CH may be driven by formation of the hydrophobic cluster and R983-D985′ salt bridges (FIG. 12). The released energy may compensate for the disruption of interactions with S1 as the S2 core rotates and becomes more compact (FIG. 3). Thus, the SRL-mediated lengthening of the CH coiled-coil may precede, and even facilitate, S1 shedding by releasing energy via relaxation of multiple strained structural elements.

Why are these changes not seen in the current open-RBD experimental structures? Each HTH motif connecting CH and HR1 is covered by two different RBDs (FIG. 8). This suggests that a single open RBD is insufficient to trigger changes in CH; requiring multiple open RBDs could have a functional importance by preventing spurious spike activation when a single RBD opens to search for a target receptor. Only when a single RBD is held open for significant periods (such as by ACE2 binding) does it become likely for a second RBD to open (6, 34). Experimental structures such as 7CAK display multiple open, bound RBDs, however it is plausible that stabilization of the top of the CH via the 2P substitution also suppresses the motion of the upper HR1. Fewer structures have been solved for the wild-type pre-fusion spike (16 of 80 in the data set); all are either closed or 1-up. One study observed the 2-up unbound wild-type spike on inactivated virions, but upon purification only the closed form was present and a wild-type 2-up structure was not refined (15).

Even with open RBDs, S2 triggering may be slow due to the need to disrupt the HTH hydrophobic cluster and CH N-cap, leading to a kinetically controlled process as in hemagglutinin (46). pH also has been suggested to play a role in RBD opening (47). pH may also be a factor in the kinetics of CH extension; protonation of Asp weakens N-capping and can give rise to pH-dependent protein conformational changes (48). The D985 N-cap may be particularly sensitive due to an expected upward shift in pKa from the close proximity of E988 (FIG. 2). Overall, the role of pH in spike-mediated membrane fusion remains to be elucidated.

Is it reasonable for these changes in S2 to take place prior to S1 shedding? A confounding factor in the analyses presented above is that the experimental pre-fusion and post-fusion structures have many other differences, primarily the presence or absence of the entire S1 subunit. It remains unclear if a partially extended coiled-coil would fit under open RBDs. If so, is the length of the SRL sufficient to retain the link to helix-3 in HR1 as the coiled-coil becomes more distant? Where could the unfolded SRL be accommodated? Furthermore, would the coiled-coil and new salt bridges be able to form within the constraints imposed by the S1 shell? Since the currently available experimental structures do not address these questions, existing knowledge is supplemented here using model building and all-atom simulations.

Simulation Results

Standard MD simulations of the closed and 3-up spike ectodomain. Following protocols in previous work (49), models were prepared of the wild-type spike glycoprotein in explicit water in 2 conformations: with all three RBDs either closed (based on 6XR8) or open (based on 7CAK). Four independent, unrestrained simulations of ˜400 ns were carried out for each conformation. Both systems were reasonably stable, and no significant changes in the top of S2 or the SRL were observed (RMSD values of 0.5-1.5 Å compared to the 6XR8 closed spike, FIG. 15).

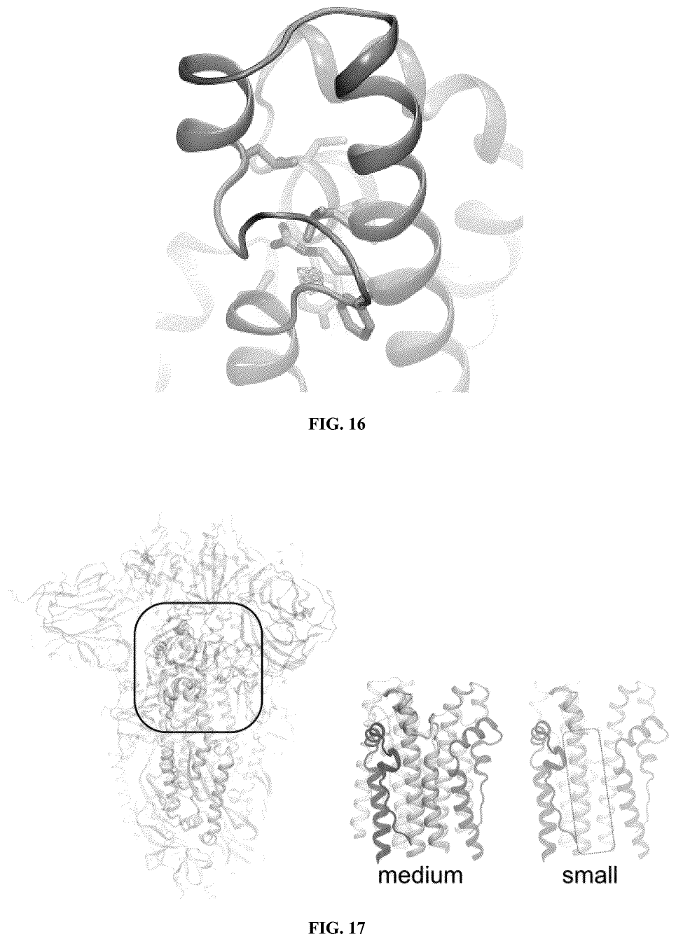

In all simulations, a water molecule diffused from the bulk solvent into the hydrophobic pocket under each SRL, bridging the R1000 NE and the backbone O of 5968; water density analysis confirmed localization of water at this position in all three protomers (FIG. 16). No change in local geometry was needed, suggesting that this site may be occupied by a water molecule in cryo-EM experiments. This is consistent with reports that arginine in nonpolar environments often retains contact to water microdroplets; the affinity of the first water molecule to guanidinium has been estimated to be 10-11 kcal/mol (50).

Model systems probe longer timescale dynamics of S2. Following the pioneering work of Carr and Kim in which peptide fragments were used to reveal the spring-loaded mechanism of hemagglutinin (28), two smaller model systems were created using different subsets of the pre-fusion S2 subunit (shown in FIG. 17). In the “medium” system, the central components of the trimer were retained, including the CH, SRL, HR1 and UH (M731-K776, D950-R1019). All amino acids below the fulcrum of S2 rotation (FIG. 14) were removed, and neutral termini were added and restrained during MD. In the “small” model, we further truncated the medium system by deleting the SRL and lower HR1, from D950 to S975, with neutral termini added but no restraints applied to V976. Both systems were simulated in full atomic detail with explicit water.

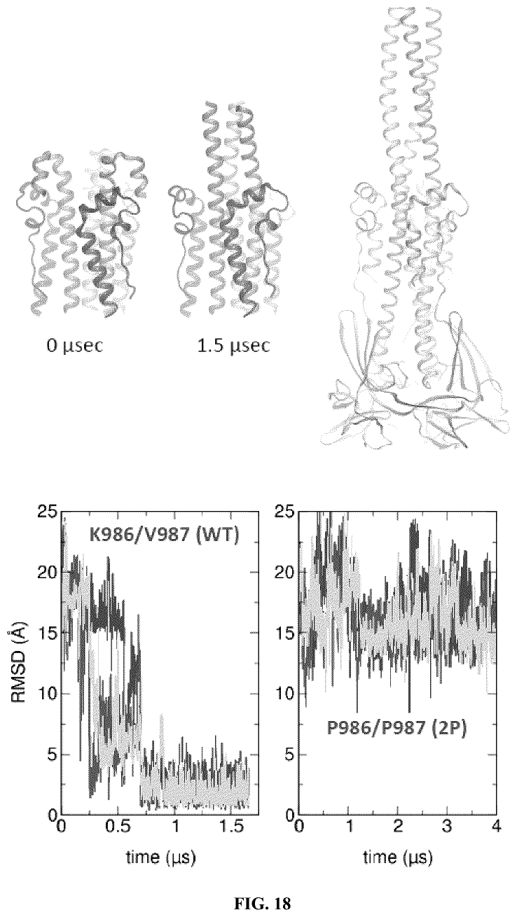

Simulations of the small model were begun in order to determine if a single α-helix is the preferred conformation of the HTH motif. During simulations of 1.5 pec in explicit water for the wild-type sequence, the HR1 helix-4 in all three protomers spontaneously rotated upwards to form a continuous helix with CH. The final structure closely matches the conformation of the same region in the post-fusion spike 6XRA, with RMSD values decreasing from the initial ˜18 Å to ˜2 Å (FIG. 18). These results provide evidence that the pre-fusion helix-turn-helix motif at the top of the S2 subunit is locally strained, and in the absence of the SRL, the system spontaneously relaxes the HTH to a structure in which CH and HR1 form a continuous α-helix. This shows strong similarity to the spring-loaded mechanism (51) in hemagglutinin.

Importantly, control simulations of the same model system using the 2P substitution showed no extension of the CH in any of the protomers, with RMSD values remaining near 15 Å during 4 pec MD (FIG. 18). This dramatically contrasts with the results obtained with the wild-type model, supporting our hypothesis that the 2P spike may be hindered from sampling the HR1 rotation.

It was asked next whether the SRL, once unfolded, could serve as a linker from HR1 helix-3 to the top of the extended CH. Initial MD simulations of the medium model were run for 400 ns, in which the pre-fusion structure was stable and no upward rotation of the short HR1 helix was observed. The difference from the small model MD suggests that unfolding the SRL, or perhaps breaking the hydrogen bond between R1000 and 5975 (discussed above) may contribute a significant kinetic barrier; therefore, we used steered molecular dynamics (SMD) to achieve CH extension (See, Methods). Over the course of a 40 ns simulation, the α-carbon atoms of each helix-turn-helix (using N978-E1017) were steered to match the structure for this region obtained from MD on the small model. The SRL (5967-L977) was allowed to move freely, and the remainder of the system was weakly restrained to the initial post-fusion structure. After SMD, an additional 1 pec of MD was carried out without any restraints except at the truncated base; the extended helices were stable during this simulation. The final structure is shown in FIG. 19.

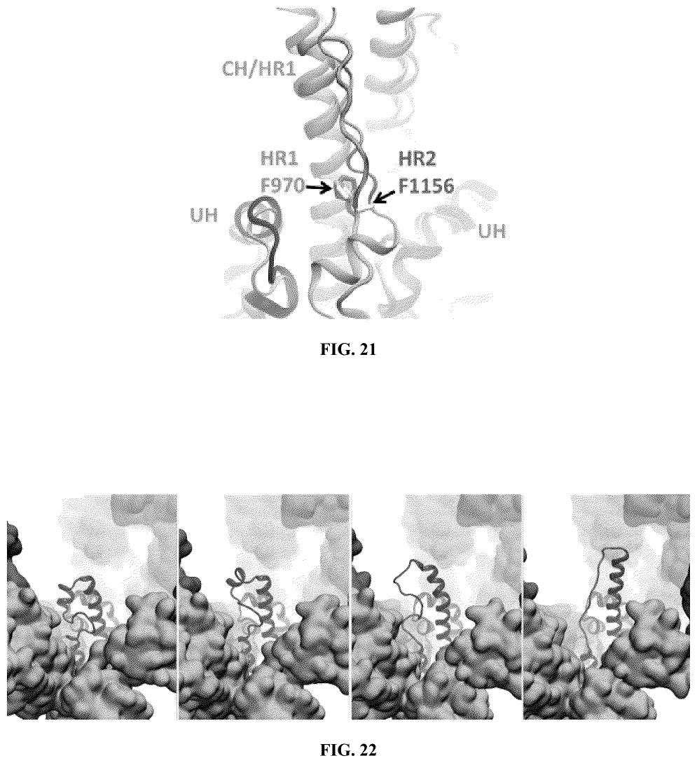

In the pre-fusion structure, the CH is N-capped by D985, with hydrophobic side chains on HR1 packing against CH (FIG. 2). After extension of CH in the medium model, a highly similar N-capping motif formed, including hydrogen bonding from the side chain of N978 to the exposed backbone NH of I980/L981 at the new helix N-terminal, and the hydrophobic V976 and/or L977 stabilizing the turn leading down the SRL to HR1 (FIG. 20). The lower HR1 helix remains connected to the top of the extended CH via unfolding of the SRL (FIG. 20). The placement of the unfolded SRL alongside the CH in the model here, is comparable to an extended segment of HR2 that follows the same path in the post-fusion structure, with F970 in a similar location as F1156 in the post-fusion spike (FIG. 21).

Simulations of CH extension using the full spike ectodomain. The S2-only model systems cannot provide insight into whether the observed process of SRL unfolding and CH extension can take place prior to S1 shedding, where it must occur inside the steric constraints of the 50 Å-wide (6) cylinder formed by the NTDs and open RBDs of S1 (FIG. 2). No significant changes to S2 were seen in the 3-up standard MD simulations described above, so the 3-up pre-fusion spike was steered to adopt the final conformation of the SRL and HTH motif that were obtained from the medium model system. Next, a pathway was refined for CH extension in the full spike by using the nudged elastic band method (NEB), with a protocol similar to what was employed (49) to map the RBD opening pathway. Here, the endpoints were defined as the 3-up spike before and after CH extension. It was not aimed at this time to find a globally optimal path revealing the exact order of events for such a complex change, but simply to ensure that a reasonable transition pathway was possible without significant steric clash with S1. Snapshots during CH extension are shown in FIG. 22; the short HR1 helix rotates upward as the SRL unfolds. The NEB pathway mapping was followed by a fully unrestrained, 250 ns MD simulation on the spike with extended CH.

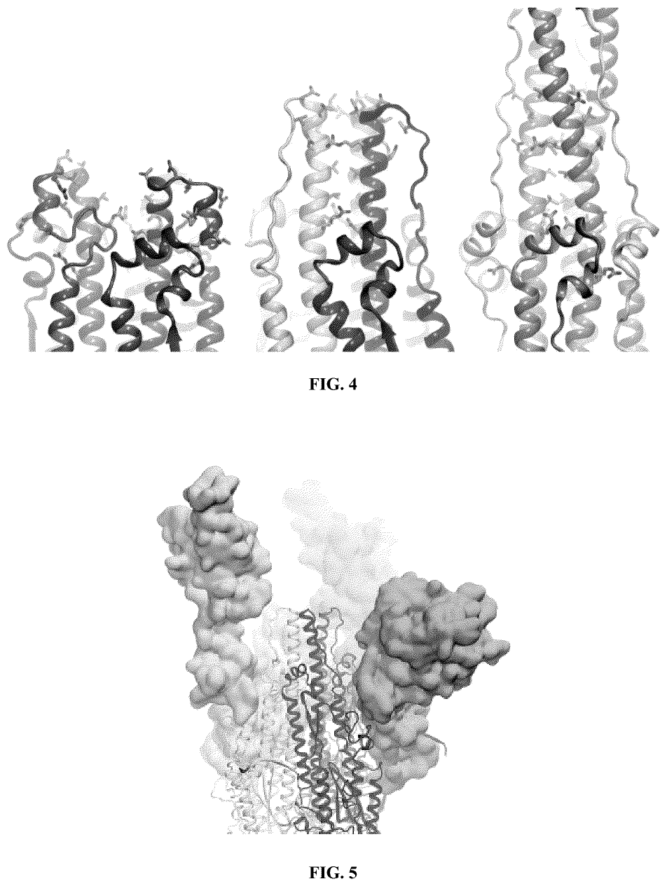

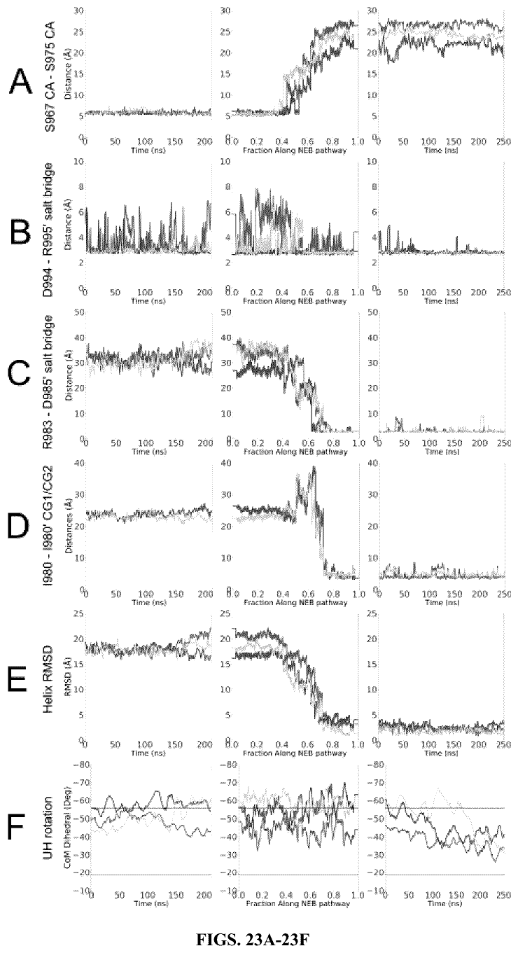

As with the model systems, the RMSD of the HTH compared to the post-fusion spike was reduced from ˜18 Å to ˜3 Å as the SRL unfolded, where it remained during unrestrained MD (time traces shown in FIGS. 23A, 23E). N978 forms an N-capping interaction with the CH in all three protomers, similar to the N-capping motif involving D985 in the pre-fusion structure. The hydrophobic cluster of I980/L984 side chains stabilizing the coiled-coil, and salt bridges between R983′/D985′ (FIG. 4) also formed as the CH lengthened (FIGS. 23B, 23D). Prior to CH extension, the D994/R995′ pairs sampled a broad distance distribution similar to that in the pre-fusion PDB structures (FIG. 11L), but closer distances corresponding to stable salt bridges were observed after unfolding of the SRL (FIG. 23B).

The upper portion of the S2 subunit in the intermediate state model is compared to the same region in the pre-fusion and post-fusion cryo-EM structures in FIG. 4, and the intermediate structure including the S1 subunit with RBDs is shown in FIG. 5. The comparison highlights the role of SRL unfolding in allowing the HR1 helix-4 to extend the CH, while maintaining a connection to the HR1 helix-3 that is held in place by S1.

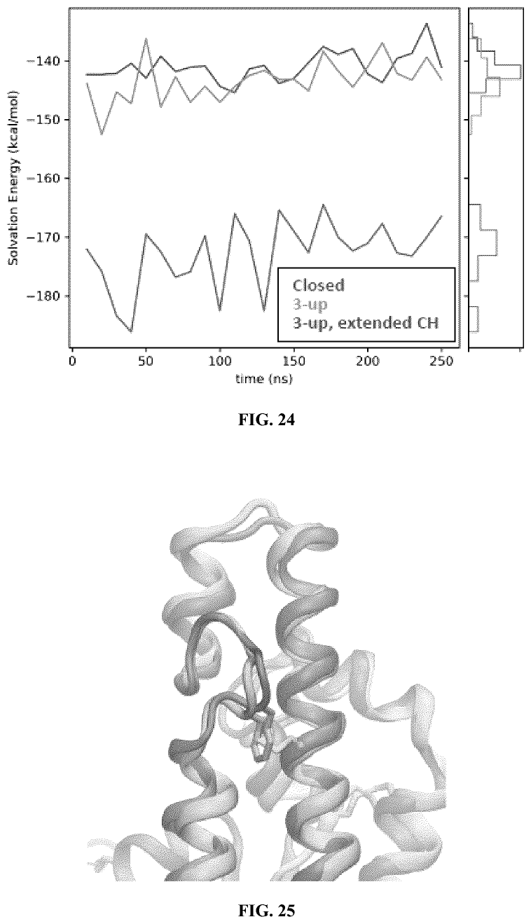

Importantly, R1000 is exposed to solvent upon SRL unfolding. We estimated the hydration free energy change of R1000 by applying a Poisson-Boltzmann (PB) approach to MD snapshots, with average per-protomer solvation free energies of −47±1, −48±1 and −58±2 kcal/mol in the closed, 3-up, and 3-up extended-CH spike respectively (FIG. 24). The data indicate that RBD opening does little to mitigate the poor environment of R1000, but significant energy is released upon SRL unfolding (˜10 kcal/mol per protomer). Exposing this buried cation to solvent may provide a significant energetic contribution to the “spring-loading” of the spike.

A notable difference in the pre-fusion and post-fusion spike is the rotation of the upper S2 relative to lower S2, presumably driven by relaxation in S2 packing once the SRL is removed from the wedge position between CH and UH (FIGS. 3, 14). The rotation was quantified using a dihedral angle for each UH; the angle is relatively stable in MD for the 3-up pre-fusion spike, but after SRL unfolding and extension of the CH, the UH slowly approach (but did not yet reach) the rotation adopted in the post-fusion spike (FIG. 23F). This suggests that even the small change of extending the CH by two turns is sufficient to trigger more extensive rearrangements in S2 despite the constraints imposed by S1. A weakened S1-S2 interface may be readily disrupted by lateral forces on the S1 subunit when both spike and ACE2 are bound to their respective membranes (17), leading to S1 shedding. Future simulations on longer timescales may provide additional insight into the destabilization of the S1-S2 interface.

The structural model suggests a possible experimental approach to probing the role of the SRL. F970 and G999 are favorably positioned to form a disulfide bond connecting the lower part of the SRL to the CH. We performed standard MD simulations of the F970C/G999C pre-fusion spike, and the disulfide bond was accommodated with minimal structure perturbation (FIG. 25). In the context of ACE2 binding, a non-2P spike with the F970C/G999C disulfide may allow the observation of early changes in the HTH motif that are frustrated in the 2P spike, while still preventing the dissociation of the lower HR1 that is required for the complete conversion to the post-fusion structure.

### CONCLUSIONS

Based on data extracted from 81 experimental spike structures in the PDB, along with observations from all-atom simulations, a mechanistic hypothesis was proposed for receptor-induced triggering of the S2 fusion machinery. A key component is the SRL, an unusual loop that is ubiquitous in coronavirus spike structures. The model rationalizes the compact structure of the SRL, its placement between two otherwise continuous α-helical segments in HR1, and the roles of several nearby highly conserved amino acids. Upon receptor binding, unfolding of the SRL and upward rotation of the HR1 can extend the CH by an additional two turns of α-helix, with the extended SRL retaining the link between the longer helix and the HR1 that remains held in place by the CTD1 of the S1 subunit. This process may be driven by spike folding energy that is stored in highly strained structure elements near the SRL that relax following SRL unfolding. The simulations suggest that commonly used stabilizing substitutions at the top of CH may hinder these conformational changes, providing a possible rationale for why they have not been observed in experimental spike structures.

The analysis provides evidence of a model for how the fusion core can be activated directly by binding of the spike to host receptors such as ACE2. A more detailed mechanism will facilitate the rational design of small molecules that could block these changes, or perhaps serve as a catalyst to trigger them prematurely, leading to irreversible activation of the spike and neutralization of the virus. The highly conserved fusion mechanism suggests that such approaches could have broad applicability to coronaviruses.

Data generated based on the model herein demonstrate that vaccines containing prefusion-stabilizing S mutations elicit antibody responses in humans with enhanced recognition of S and the S1 subunit relative to postfusion S as compared with vaccines lacking these mutations or natural infection. Prefusion S and S1 antibody binding titers positively and equivalently correlated with neutralizing activity, and depletion of S1-directed antibodies completely abrogated plasma neutralizing activity. See, Bowen and Veesler et al., Sci. Immunol. 7, eadf1421 (2022) 23 Dec. 2022, incorporated herein in its entirety.

### REFERENCES

1. Y. Watanabe, J. D. Allen, D. Wrapp, J. S. McLellan, M. Crispin, Site-specific glycan analysis of the SARS-CoV-2 spike. Science 369, 330-333 (2020).
2. H. Zhang, J. M. Penninger, Y. Li, N. Zhong, A. S. Slutsky, Angiotensin-converting enzyme 2 (ACE2) as a SARS-CoV-2 receptor: molecular mechanisms and potential therapeutic target. Intensive Care Medicine 46, 586-590 (2020).
3. J. Shang et al., Structural basis of receptor recognition by SARS-CoV-2. Nature 581, 221-224 (2020).
4. M. Hoffmann et al., SARS-CoV-2 Cell Entry Depends on ACE2 and TMPRSS2 and Is Blocked by a Clinically Proven Protease Inhibitor. Cell 181, 271-280.e278 (2020).
5. J. Shang et al., Cell entry mechanisms of SARS-CoV-2. Proceedings of the National Academy of Sciences 117, 11727-11734 (2020).
6. D. J. Benton et al., Receptor binding and priming of the spike protein of SARS-CoV-2 for membrane fusion. Nature 588, 327-330 (2020).
7. Q. Wang et al., Structural and Functional Basis of SARS-CoV-2 Entry by Using Human ACE2. Cell 181, 894-904.e899 (2020).
8. R. Yan et al., Structural basis for the recognition of the SARS-CoV-2 by full-length human ACE2. Science (New York, N.Y.) 2, 1444-1448 (2020).
9. J. Lan et al., Structure of the SARS-CoV-2 spike receptor-binding domain bound to the ACE2 receptor. Nature 581, 215-220 (2020).
10. Y. Huang, C. Yang, X.-f. Xu, W. Xu, S.-w. Liu, Structural and functional properties of SARS-CoV-2 spike protein: potential antivirus drug development for COVID-19. Acta Pharmacologica Sinica 41, 1141-1149 (2020).
11. D. Wrapp et al., Cryo-EM structure of the 2019-nCoV spike in the prefusion conformation. Science (New York, N.Y.) 1263, 1260-1263 (2020).
12. A. C. Walls et al., Structure, Function, and Antigenicity of the SARS-CoV-2 Spike Glycoprotein. Cell 181, 281-292 (2020).
13. W. Tai et al., Characterization of the receptor-binding domain (RBD) of 2019 novel coronavirus: implication for development of RBD protein as a viral attachment inhibitor and vaccine. Cellular & Molecular Immunology 17, 613-620 (2020).
14. M. Gui et al., Cryo-electron microscopy structures of the SARS-CoV spike glycoprotein reveal a prerequisite conformational state for receptor binding. Cell Research 27, 119-129 (2017).
15. Z. Ke et al., Structures and distributions of SARS-CoV-2 spike proteins on intact virions. Nature 588, 498-502 (2020).
16. W. Song, M. Gui, X. Wang, Y. Xiang, Cryo-EM structure of the SARS coronavirus spike glycoprotein in complex with its host cell receptor ACE2. PLoS Pathog 14, e1007236 (2018).
17. E. P. Barros et al., The flexibility of ACE2 in the context of SARS-CoV-2 infection. Biophys J 120, 1072-1084 (2021).
18. F. Li, Structure, Function, and Evolution of Coronavirus Spike Proteins. Annual Rev Virol 3, 237-261 (2016).
19. Y. Cai et al., Distinct conformational states of SARS-CoV-2 spike protein. Science 369, 1586-1592 (2020).
20. S. Belouzard, V. C. Chu, G. R. Whittaker, Activation of the SARS coronavirus spike protein via sequential proteolytic cleavage at two distinct sites. Proceedings of the National Academy of Sciences of the United States of America 106, 5871-5876 (2009).
21. B. Coutard et al., The spike glycoprotein of the new coronavirus 2019-nCoV contains a furin-like cleavage site absent in CoV of the same clade. Antiviral Research 176, 104742-104742 (2020).
22. J. A. Jaimes, N. M. André, J. S. Chappie, J. K. Millet, G. R. Whittaker, Phylogenetic Analysis and Structural Modeling of SARS-CoV-2 Spike Protein Reveals an Evolutionary Distinct and Proteolytically Sensitive Activation Loop. J Mol Biol 432, 3309-3325 (2020).
23. T. Tang, M. Bidon, J. A. Jaimes, G. R. Whittaker, S. Daniel, Coronavirus membrane fusion mechanism offers a potential target for antiviral development. Antiviral Research 178, 104792 (2020).
24. M. C. Johnson et al., Optimized Pseudotyping Conditions for the SARS-COV-2 Spike Glycoprotein. Journal of Virology 94, e01062-01020 (2020).
25. L. M. Reinke et al., Different residues in the SARS-CoV spike protein determine cleavage and activation by the host cell protease TMPRSS2. PLOS ONE 12, e0179177 (2017).
26. M. Hoffmann, H. Kleine-Weber, S. Pöhlmann, A Multibasic Cleavage Site in the Spike Protein of SARS-CoV-2 Is Essential for Infection of Human Lung Cells. Molecular Cell 78, 779-784.e775 (2020).
27. J. K. Millet, G. R. Whittaker, Physiological and molecular triggers for SARS-CoV membrane fusion and entry into host cells. Virology 517, 3-8 (2018).
28. C. M. Carr, P. S. Kim, A spring-loaded mechanism for the conformational change of influenza hemagglutinin. Cell 73, 823-832 (1993).
29. A. C. Walls et al., Tectonic conformational changes of a coronavirus spike glycoprotein promote membrane fusion. Proc Natl Acad Sci USA 114, 11157-11162 (2017).
30. C. Liu et al., Viral Architecture of SARS-CoV-2 with Post-Fusion Spike Revealed by Cryo-EM. bioRxiv 10.1101/2020.03.02.972927, 2020.2003.2002.972927 (2020).
31. C. Liu et al., The Architecture of Inactivated SARS-CoV-2 with Postfusion Spikes Revealed by Cryo-EM and Cryo-ET. Structure 28, 1218-1224.e1214 (2020).
32. I. G. Madu, S. L. Roth, S. Belouzard, G. R. Whittaker, Characterization of a Highly Conserved Domain within the Severe Acute Respiratory Syndrome Coronavirus Spike Protein S2 Domain with Characteristics of a Viral Fusion Peptide. Journal of Virology 83, 7411-7421 (2009).
33. S. Xia et al., Fusion mechanism of 2019-nCoV and fusion inhibitors targeting HR1 domain in spike protein. Cell Mol Immunol 10.1038/s41423-020-0374-2 (2020).
34. A. C. Walls et al., Unexpected Receptor Functional Mimicry Elucidates Activation of Coronavirus Fusion. Cell 176, 1026-1039 e1015 (2019).
35. T. M. Clausen et al., SARS-CoV-2 Infection Depends on Cellular Heparan Sulfate and ACE2. Cell 183, 1043-1057.e1015 (2020).
36. L. Cantuti-Castelvetri et al., Neuropilin-1 facilitates SARS-CoV-2 cell entry and infectivity. Science 370, 856 (2020).
37. J. L. Daly et al., Neuropilin-1 is a host factor for SARS-CoV-2 infection. Science 370, 861 (2020).
38. Z. Lv et al., Structural basis for neutralization of SARS-CoV-2 and SARS-CoV by a potent therapeutic antibody. Science 369, 1505 (2020).
39. J. Pallesen et al., Immunogenicity and structures of a rationally designed prefusion MERS-CoV spike antigen. Proceedings of the National Academy of Sciences 114, E7348-E7357 (2017).
40. L. Serrano, A. R. Fersht, Capping and α-helix stability. Nature 342, 296-299 (1989).
41. M. Shapovalov, S. Vucetic, R. L. Dunbrack, Jr., A new clustering and nomenclature for beta turns derived from high-resolution protein structures. PLOS Computational Biology 15, e1006844 (2019).
42. C. A. Fitch, G. Platzer, M. Okon, B. Garcia-Moreno E, L. P. McIntosh, Arginine: Its pKa value revisited. Protein Science 24, 752-761 (2015).
43. B. Roux, Lonely Arginine Seeks Friendly Environment. Journal of General Physiology 130, 233-236 (2007).
44. M. J. Harms, J. L. Schlessman, G. R. Sue, B. Garcia-Moreno E, Arginine residues at internal positions in a protein are always charged. Proceedings of the National Academy of Sciences 108, 18954 (2011).
45. X. Fan, D. Cao, L. Kong, X. Zhang, Cryo-EM analysis of the post-fusion structure of the SARS-CoV spike glycoprotein. Nature Communications 11, 3618 (2020).
46. D. Baker, D. A. Agard, Influenza hemagglutinin: kinetic control of protein function. Structure 2, 907-910 (1994).
47. T. Zhou et al., Cryo-EM Structures of SARS-CoV-2 Spike without and with ACE2 Reveal a pH-Dependent Switch to Mediate Endosomal Positioning of Receptor-Binding Domains. Cell Host & Microbe 28, 867-879.e865 (2020).
48. Y. Huang et al., Helix N-Cap Residues Drive the Acid Unfolding That Is Essential in the Action of the Toxin Colicin A. Biochemistry 58, 4882-4892 (2019).
49. L. Fallon et al., Free Energy Landscapes for RBD Opening in SARS-CoV-2 Spike Glycoprotein Simulations Suggest Key Interactions and a Potentially Druggable Allosteric Pocket. ChemRxiv Preprint (2020).
50. B. Gao, T. Wyttenbach, M. T. Bowers, Protonated Arginine and Protonated Lysine: Hydration and Its Effect on the Stability of Salt-Bridge Structures. The Journal of Physical Chemistry B 113, 9995-10000 (2009).
51. C. M. Carr, C. Chaudhry, P. S. Kim, Influenza hemagglutinin is spring-loaded by a metastable native conformation. Proceedings of the National Academy of Sciences 94, 14306 (1997).

### OTHER EMBODIMENTS

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed:

1. An engineered peptide comprising a coronavirus S1/S2 prefusion spike peptide sequence having two or more amino acid substitutions, wherein the two amino acids are substituted at conserved amino acid positions of the coronavirus S1/S2 prefusion spike peptide sequence.

2. The engineered peptide of claim 1, wherein the two amino acids comprise cysteine.

3. The engineered peptide of claim 1, wherein the coronavirus S1/S2 prefusion spike peptide comprises peptide having a 90% sequence identity to S₉₆₇SNF₉₇₀GAISSVLNDILSRLDKVEAEVQIDRLITG₉₉₉R₁₀₀₀ (SEQ ID NO: 1).

4. The engineered peptide of claim 1, wherein the coronavirus S1/S2 prefusion spike peptide comprises an amino acid sequence comprising S₉₆₇SNF₉₇₀GAISSVLNDILSRLDKVEAEVQIDRLITG₉₉₉R₁₀₀₀ (SEQ ID NO: 1).

5. The engineered peptide of claim 4, wherein the coronavirus S1/S2 prefusion spike peptide sequence comprises a cysteine substitution at amino acid position 970.

6. The engineered peptide of claim 4, wherein the coronavirus S1/S2 prefusion spike peptide sequence comprises a cysteine substitution at amino acid position 999.

7. The engineered peptide of claim 4, wherein the coronavirus S1/S2 prefusion spike peptide sequence comprises a cysteine substitution at amino acid positions 970 and 999.

8. A vaccine comprising an immunogenic peptide comprising a 90% sequence identity to S₉₆₇SNF₉₇₀GAISSVLNDILSRLDKVEAEVQIDRLITG₉₉₉R₁₀₀₀ (SEQ ID NO: 1).

9. The vaccine of claim 8, wherein the immunogenic peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1).

10. The vaccine of claim 9, wherein phenylalanine at position 970 (F970) and glycine at position 999 (G999) of SEQ ID NO: 1 are substituted with cysteines, wherein the cysteines at amino acid positions 970 and 999 form a disulfide bridge.

11. An expression vector encoding a coronavirus S1/S2 prefusion spike peptide comprising a 90% sequence identity to S₉₆₇SNF₉₇₀GAISSVLNDILSRLDKVEAEVQIDRLITG₉₉₉R₁₀₀₀ (SEQ ID NO: 1).

12. The expression vector of claim 11, wherein the coronavirus S1/S2 prefusion spike peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1).

13. The expression vector of claim 13, wherein phenylalanine at position 970 (F970) and glycine at position 999 (G999) of SEQ ID NO: 1 are substituted with cysteines and form a disulfide bridge.

14. A host cell comprising an expression vector encoding a coronavirus S1/S2 prefusion spike peptide comprising a 90% sequence identity to S₉₆₇SNF₉₇₀GAISSVLNDILSRLDKVEAEVQIDRLITG₉₉₉R₁₀₀₀ (SEQ ID NO: 1).

15. The host cell of claim 14, wherein the coronavirus S1/S2 prefusion spike peptide comprises cysteine substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1).

16. The host cell of claim 14, wherein the host cell comprises an autologous cell, an allogeneic cell, a haplotype matched cell, a haplotype mismatched cell, a haplo-identical cell, a xenogeneic cell, stem cells, cell lines, immune system cells or combinations thereof.

17. A method of preventing infection and treating a subject infected with a coronavirus, comprising administering to the subject a pharmaceutical composition comprising a therapeutically effective amount of a coronavirus S1/S2 prefusion spike peptide or expression vector encoding the coronavirus S1/S2 prefusion spike peptide.

18. The method of claim 17, wherein the coronavirus S1/S2 prefusion spike peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1).

19. The method of claim 17, further comprising administering to the subject an agent or vaccine.

20. The method of claim 19, wherein the agent comprises an anti-viral agent, an immunomodulatory agent, an antibody, an antibody fragment, a chemotherapeutic agent, or a biological agent.

21. An engineered peptide comprising an amino acid sequence having a 90% amino acid sequence identity to S₉₆₇SNF₉₇₀GAISSVLNDILSRLDKVEAEVQIDRLITG₉₉₉R₁₀₀₀ (SEQ ID NO: 1), wherein the peptide is conjugated to a secondary agent.

22. The engineered peptide of claim 21, wherein the engineered peptide comprises at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1).

23. A composition comprising two or more conjugated peptides wherein the peptides comprise an amino acid sequence having a 90% amino acid sequence identity to S₉₆₇SNF₉₇₀GAISSVLNDILSRLDKVEAEVQIDRLITG₉₉₉R₁₀₀₀ (SEQ ID NO: 1).

24. The composition of claim 23, wherein the two or more conjugated peptides comprise at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1).

25. The composition of claim 24, wherein the two or more peptides are conjugated via a linker molecule.

26. The composition of claim 24, wherein the two or more peptides are fused to each other.

27. The composition of claim 23, further comprising an adjuvant.

28. A nucleic acid molecule comprising a nucleotide sequence encoding an amino acid sequence having a 90% amino acid sequence identity to S₉₆₇SNF₉₇₀GAISSVLNDILSRLDKVEAEVQIDRLITG₉₉₉R₁₀₀₀ (SEQ ID NO: 1).

29. A method of identifying candidate therapeutic agents for preventing or treating a coronavirus infection comprising contacting a substrate with:
(i) a nucleic acid molecule encoding a peptide having a 90% amino acid sequence identity to S₉₆₇SNF₉₇₀GAISSVLNDILSRLDKVEAEVQIDRLITG₉₉₉R₁₀₀₀ (SEQ ID NO: 1); or,
(ii) a peptide having a 90% amino acid sequence identity to S₉₆₇SNF₉₇₀GAISSVLNDILSRLDKVEAEVQIDRLITG₉₉₉R₁₀₀₀ (SEQ ID NO: 1); or,
(iii) a nucleic acid molecule encoding a peptide comprising at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1), with a candidate therapeutic agent; or,
(iv) a peptide comprising at least two amino acid substitutions at amino acid positions 970 and 999 of the amino acid sequence comprising S₉₆₇SNF₉₇₀GAISSVLNDILSRLDKVEAEVQIDRLITG₉₉₉R₁₀₀₀ (SEQ ID NO: 1), with a candidate therapeutic agent; or,
(v) a cell comprising any one of nucleic acids or peptides of (i)-(iv);
and
conducting an assay for measuring an output value.

30. The method of claim 29, wherein the assay comprises: immunoassays, Southern blots, Western blots, polymerase chain reaction (PCR), Northern blots, sequencing, reverse-transcriptase PCR, microarray technology, immunohistochemistry, enzyme-linked immunosorbent assay, flow cytometry mass spectrometry, Förster resonance energy transfer, time-resolved fluorescence energy transfer, amplified luminescent proximity homogeneous assay, fluorescence polarization, cell based assays or combinations thereof.

31. The method of claim 30, wherein the assay is a high throughput screening (HTS) assay.

32. A coronavirus S1/S2 prefusion spike protein comprising a disulfide bridge between a cysteine substituted for a phenylalanine (Phe) located between at least amino acid position 800 to about amino acid position 1100 and a cysteine substituted for a glycine (Gly) located 29 amino acids from the Phe amino acid, wherein the cysteines form a disulfide bridge.

33. The coronavirus S1/S2 prefusion spike protein of claim 32, wherein a Phe at amino acid position 970 and a Gly at amino acid position 999 are each substituted with a cysteine.

34. The coronavirus S1/S2 prefusion spike protein of claim 32, wherein a Phe at amino acid position 1060 and a Gly at amino acid position 1089 are each substituted with a cysteine.

35. The coronavirus S1/S2 prefusion spike protein of claim 32, wherein a Phe at amino acid position 1036 and a Gly at amino acid position 1065 are each substituted with a cysteine.

36. The coronavirus S1/S2 prefusion spike protein of claim 32, wherein a Phe at amino acid position 1044 and a Gly at amino acid position 1073 are each substituted with a cysteine.

37. The coronavirus S1/S2 prefusion spike protein of claim 32, wherein a Phe at amino acid position 952 and a Gly at amino acid position 981 are each substituted with a cysteine.

38. The coronavirus S1/S2 prefusion spike protein of claim 32, wherein a Phe at amino acid position 839 and a Gly at amino acid position 868 are each substituted with a cysteine.

39. The coronavirus S1/S2 prefusion spike protein of claim 32, wherein a Phe at amino acid position 843 and a Gly at amino acid position 872 are each substituted with a cysteine.

40. The coronavirus S1/S2 prefusion spike protein of claim 32, wherein a Phe at amino acid position 1064 and a Gly at amino acid position 1093 are each substituted with a cysteine.

41. The coronavirus S1/S2 prefusion spike protein of claim 32, wherein a Phe at amino acid position 1020 and a Gly at amino acid position 1049 are each substituted with a cysteine.

42. A coronavirus spike protein comprising an F to C (Phe to Cys) substitution and a G to C (Gly to Cys) substitution at positions corresponding to F970 and G999 of SARS-CoV-2 spike protein.
